(19) United States
(12) Patent Application Publication
(10) Pub. No.: US 2024/0366752 A1
(43) Pub. Date: Nov. 7, 2024

(54) VACCINE COMPOSITION FOR PREVENTION AGAINST COVID-19

(71) Applicant: KOREA ADVANCED INSTITUTE OF SCIENCE AND TECHNOLOGY, Daejeon (KR)

(72) Inventors: Heung Kyu LEE, Daejeon (KR); Hi Eun JUNG, Daejeon (KR); Keun Bon KU, Daejeon (KR)

(21) Appl. No.: 18/686,928

(22) PCT Filed: Aug. 29, 2022
(86) PCT No.: PCT/KR2022/012889

§ 371 Date: Feb. 27, 2024

(30) Foreign Application Priority Data
Aug. 27, 2021 (KR) 10-2021-0113976

(51) Int. Cl. A61K 39/215; A61K 39/00; A61K 39/39; A61P 37/04; C12N 15/86

(52) U.S. Cl. CPC A61K 39/215; A61K 39/39; A61P 37/04; C12N 15/86; A61K 2039/5256; A61K 2039/541; A61K 2039/543; A61K 2039/55522; C12N 2710/10043; C12N 2770/20034

(57) ABSTRACT

The present invention relates to a vaccine composition for preventing or treating coronavirus disease (COVID-19) comprising a recombinant adenovirus as an active ingredient. The present invention enhances immune responses against severe acute respiratory syndrome coronavirus 2 (SARS-CoV-2), which is a severe pandemic that has resulted in millions of deaths worldwide, through the recombinant adenovirus, and thus may be useful as a prophylactic vaccine composition that provides fundamental and efficient protection against SARS-CoV-2.

### CROSS-REFERENCE TO RELATED APPLICATIONS

This application is a U.S. National Stage Entry of International Patent Application no. PCT/KR2022/012889, filed Aug. 29, 2022, which claims the benefit of priority of Korean Patent Application no. 10-2021-0113976, filed Aug. 27, 2021.

### REFERENCE TO AN ELECTRONIC SEQUENCE LISTING

This application contains a Sequence Listing which has been submitted electronically and is hereby incorporated by reference in its entirety. The Sequence Listing was created on Feb. 26, 2024, is named “24-0272-WO-US_SequenceListing_ST26” and is 19,386 bytes in size.

### TECHNICAL FIELD

The present invention relates to a vaccine composition for preventing novel coronavirus infection.

### BACKGROUND ART

Severe acute respiratory syndrome coronavirus 2 (SARS-CoV-2), a novel virus that causes coronavirus disease 2019 (COVID-19), is responsible for a pandemic that has resulted in millions of deaths worldwide. There is an urgent need for effective vaccines to prevent COVID-19 and eradicate SARS-CoV-2, and many companies are developing and testing new vaccines. These include not only traditional vaccine platforms, such as inactivated and attenuated viruses or subunit vaccines, but also new RNA, DNA, and viral vector vaccines. Despite advances in vaccine platform technology, research on the most effective delivery routes for vaccines is limited. Since SARS-CoV-2 infects via the respiratory tract, intranasal vaccination should be effective. However, poor understanding of mucosal vaccines has limited their development to the stage of human clinical trials.

To eradicate SARS-CoV-2 and prevent further infections, many vaccine candidates have been developed. The delivery of these vaccines is limited to intramuscular vaccination, in contrast to the diversity in the platform technology. Although intramuscular vaccination is safe and effective, mucosal vaccination could improve the local immune responses that block the spread of pathogens. However, due to a lack of understanding of mucosal immunity combined with the urgent need for a COVID-19 vaccine, only intramuscular vaccinations have become possible.

While the angiotensin-converting enzyme (ACE2) receptor for SARS-CoV-2 is found throughout the respiratory tract and in the brain, placenta, and gut, the first line of defense against infection is the nasal epithelium. Intramuscular vaccination induces an immune response in the lower respiratory tract (LRT), but induces limited immunity in the upper respiratory tract (URT). In contrast, intranasal vaccination provides immunity not only in the URT but also provides systemic immunity. Mucosal IgA is known to protect against the shedding of nasal virus early in infection, while the level of systemic IgA is correlated with severe disease. However, mucosal immunity can be difficult to establish because the mucosa is frequently exposed to and becomes tolerant of foreign molecules. Additionally, innate mucosal defense systems such as proteolytic enzymes present a barrier to antigen absorption. For the development of effective mucosal vaccines, there is a need for a better understanding of the mucosal immune environment.

### DISCLOSURE

### Technical Problem

Severe acute respiratory syndrome coronavirus 2 (SARS-CoV-2) is responsible for a severe pandemic that has resulted in millions of deaths worldwide, and the present inventors have made extensive research efforts to develop an effective vaccine against SARS-CoV-2. As a result, the present inventors have developed a recombinant expression vector using the surface spike protein of the coronavirus and an adjuvant, and have found that the immune response against the coronavirus is enhanced by intranasal injection of the recombinant expression vector. Accordingly, the present inventors have identified a fundamental and efficient vaccine composition against SARS-CoV-2, thereby completing the present invention.

Therefore, an object of the present invention is to provide a vaccine composition for preventing coronavirus disease (COVID-19) comprising recombinant adenovirus as an active ingredient.

### Technical Solution

Hereinafter, various embodiments described herein will be described with reference to figures. In the following description, numerous specific details are set forth, such as specific configurations, compositions, and processes, etc., in order to provide a thorough understanding of the present invention. However, certain embodiments may be practiced without one or more of these specific details, or in combination with other known methods and configurations. In other instances, known processes and preparation techniques have not been described in particular detail in order to not unnecessarily obscure the present invention. Reference throughout this specification to “one embodiment” or “an embodiment” means that a particular feature, configuration, composition, or characteristic described in connection with the embodiment is included in at least one embodiment of the present invention. Thus, the appearances of the phrase “in one embodiment” or “an embodiment” in various places throughout this specification are not necessarily referring to the same embodiment of the present invention. Additionally, the particular features, configurations, compositions, or characteristics may be combined in any suitable manner in one or more embodiments.

Unless otherwise stated in the specification, all the scientific and technical terms used in the specification have the same meanings as commonly understood by those skilled in the technical field to which the present invention pertains.

According to one aspect of the present invention, the present invention provides a recombinant expression vector comprising: a gene sequence encoding the coronavirus surface spike protein (S protein) comprising a mutation at amino acid position 614 of the spike protein: an adjuvant gene sequence; and a gene sequence encoding a P2A peptide.

The present inventors have made extensive research efforts to develop an effective vaccine against SARS-CoV-2 that is a severe pandemic. As a result, the present inventors have developed a recombinant expression vector using the surface spike protein of the coronavirus and an adjuvant, and have found that the recombinant expression vector enhances the immune response against the coronavirus when injected intranasally. Accordingly, the present inventors have identified a fundamental and efficient vaccine composition against SARS-CoV-2, thereby completing the present invention.

In the present specification, the term “coronavirus” collectively refers to RNA viruses belonging to the Coronavirinae subfamily of the Coronaviridae family. Coronavirus causes respiratory and digestive system infections in humans and animals, and is easily transmitted mainly through mucous membrane infection and droplet transmission. It generally causes mild respiratory infections in humans, but can also cause fatal infections in humans, and may cause diarrhea in cattle and pigs and cause respiratory diseases in chickens.

In the present specification, the term “adjuvant” refers to a substance that acts to accelerate, prolong, or enhance an antigen-specific immune response when used in combination with a specific vaccine antigen.

In the present specification, the term “spike protein (S protein)” is also referred to as peplomer, and refers to a protruding protein that protrudes outward from the viral capsid or viral envelope that can be seen through an electron microscope. The spike protein is utilized when the virus binds to a receptor on a host cell, and is a large, highly glycosylated transmembrane fusion protein composed of 1,160 to 1,400 amino acids depending on the virus type.

In the present specification, the term “vector” refers to a means for expressing the gene of interest in a host cell. The vector may comprise elements for expression of the gene of interest, including a replication origin, a promoter, an operator, a transcription terminator, and the like, and may further comprise appropriate enzyme sites (e.g., restriction enzyme sites) for introduction of the gene of interest into the vector, and/or a selection marker for identifying successful introduction into the host cell, and/or a ribosome binding site (RBS) for translation into protein, an internal ribosome entry site (IRES), and the like. The vector may be engineered by a conventional genetic engineering method so as to have the above-described fusion polynucleotide (fusion promoter) as a promoter. The vector may further comprise a transcription regulatory sequence (e.g., an enhancer, etc.), in addition to the promoter.

In the present specification, the term “expression vector” refers to a recombinant vector capable of expressing a peptide of interest in a desired host cell, and means a gene construct that contains essential regulatory elements operably linked so that an inserted gene is expressed. The expression vector contains expression regulatory elements such as an initiation codon, a stop codon, a promoter, and an operator. The initiation codon and stop codon are generally considered as part of a nucleotide sequence encoding a polypeptide, and should exhibit action in s subject when the gene construct is administered, and should be in frame with a coding sequence. The promoter of the vector may be constitutive or inducible. The vector may be introduced into a host cell in the form of an expression cassette, which is a gene construct including all the elements necessary for self-expression. The expression cassette may usually include a promoter operably linked to the inserted gene to be expressed, a transcription termination signal, a ribosome binding site, and a translation termination signal. The expression cassette may be in the form of an expression vector capable of self-replication. In the present invention, the expression vector may be a viral or non-viral vector. The viral vector may be an adenoviral vector, a retroviral vector including a lentivirus, an adeno-associated viral vector, or a herpes simplex virus vector, without being limited thereto. In addition, the non-viral vector may be a plasmid vector, mRNA, a bacteriophage vector, a liposome, a bacterial artificial chromosome, an artificial yeast chromosome, or the like, without being limited thereto.

According to a specific embodiment of the present invention, the gene sequence encoding the spike protein (S protein) comprising a mutation at amino acid position 614 of the spike protein is SEQ ID NO: 1. Specifically, the mutation is an aspartic acid (D)-to-glycine (G) substitution.

In the present specification, the term “aspartic acid (D)” refers to one of the 20 important amino acids, which is known as aspartate, which is the anionic form of aspartic acid. Aspartic acid is a carboxylic acid analogue of asparagine and is a reaction product of the urea cycle.

In the present specification, the term “glycine (G)” refers to one of the 20 basic amino acids, which is commonly found in animal proteins. Glycine has a hydrogen (—H) as its side chain and is the smallest and simplest amino acid. Because of these properties, glycine can occupy small spaces which cannot be easily occupied by other amino acids.

According to a specific embodiment of the present invention, the adjuvant gene is a chemokine (C-X-C motif) ligand 9 (CXCL9) gene, or an interleukin 7 (IL-7) gene. Specifically, the chemokine (C-X-C motif) ligand 9 gene is set forth in SEQ ID NO: 2, and the interleukin 7 gene is set forth in SEQ ID NO: 3.

In the present specification, the term “chemokine (C-X-C motif) ligand 9 (CXCL9)” refers to a small cytokine belonging to the CXC chemokine family, which is also known as monokine induced by gamma interferon. CXCL9 plays a role in inducing chemotaxis, promoting differentiation and proliferation of leukocytes, and causing tissue extravasation.

In the present specification, the term “interleukin 7 (IL-7)” refers to the protein encoded by the IL7 gene in humans. IL-7 is a hematopoietic growth factor secreted by stromal cells of bone marrow and thymus, and is produced by keratinocytes, dendritic cells, hepatocytes, neurons, and epithelial cells, but not normal lymphocytes.

According to a specific embodiment of the present invention, the gene encoding a P2A peptide is set forth in SEQ ID NO: 5.

In the present specification, the term “P2A peptide (P2A self-cleaving peptide)” refers to one of the four members of the 2A peptide family. The P2A peptide can induce ribosome skipping during protein translation in cells.

According to another aspect of the present invention, the present invention provides a recombinant transformant transformed with the recombinant expression vector.

As used herein, the term “transformation” refers to a molecular biological phenomenon in which a DNA chain fragment or plasmid containing a gene of type different from that of the original cell penetrates among the cells to express a new genetic trait. Transformation is often observed in bacteria, and may also be achieved by artificial genetic engineering. A cell transformed by receiving a DNA that is not its own DNA is referred to as a transformed competent cell.

In the present specification, the term “transformant” refers to a cell or plant transformed with a DNA construct consisting of a DNA sequence, operably linked to a promoter and encoding a useful substance, and a recombinant protein product produced thereby. In the present invention, transformant is meant to include transformed microorganisms, animal cells, plant cells, transformed animals or plants, and cultured cells derived therefrom.

In the present invention, transfer (introduction) of the expression vector into cells may be performed using a transfer method widely known in the art. Examples of the transfer method include, but are not limited to, microinjection, calcium phosphate precipitation, electroporation, sonoporation, magnetofection using a magnetic field, liposome-mediated transfection, gene bombardment, and a method using dendrimers and inorganic nanoparticles.

According to a specific embodiment of the present invention, the transformant is selected from the group consisting of a microorganism, a cell, an animal, a plant, and a virus.

According to a specific embodiment of the present invention, the virus is an adenovirus. Specifically, the adenovirus may be adenovirus type 5, without being limited thereto.

In the present specification, the term “adenovirus” refers to a medium-sized (90 to 100 nm) virus. The adenovirus has no envelope, is icosahedral in shape, and has double-helix DNA. Viruses belonging to the adenoviridae family can infect various vertebrates, including humans, and adenoviruses were named after human adenoids, from which they were first isolated.

According to another aspect of the present invention, the present invention provides a vaccine composition for preventing coronavirus disease (COVID-19) comprising the transformant transformed with the expression vector.

In the present specification, the term “prevention” means inhibiting the occurrence of a disorder or a disease in a subject who has never been diagnosed as having the disorder or disease, but is likely to be afflicted with such disorder or disease, and includes any action that delays the growth, proliferation, invasiveness, or infectiousness of virus by administration of the composition.

In the present specification, the term “administration” or “administer” means administering a therapeutically effective amount of the composition of the present invention directly to a subject so that the same amount is formed in the subject's body.

According to the present specification, “subject” includes, without limitation, humans, mice, rats, guinea pigs, dogs, cats, horses, cows, pigs, monkeys, chimpanzees, baboons or rhesus monkeys. Specifically, the subject of the present invention is a human.

The antigenic composition or vaccine of the present invention may further comprise a solvent, an excipient, and the like. Examples of the solvent include, but are not limited, physiological saline and distilled water, and examples of the excipient include, but are not limited to, aluminum phosphate, aluminum hydroxide, and aluminum potassium sulfate. In addition, the antigenic composition or vaccine of the present invention may further comprise substances that are commonly used for vaccine production in the art to which the present invention pertains.

The antigenic composition or vaccine of the present invention may be produced by methods that are commonly used in the art to which the present invention pertains. The antigenic composition or vaccine of the present invention may be prepared as an oral or parenteral formulation, and is preferably prepared as an injectable liquid formulation which is a parenteral formulation. The antigenic composition or vaccine of the present invention may be administered through an intradermal, intramuscular, intraperitoneal, intravenous, subcutaneous, nasal or epidural route.

The antigenic composition or vaccine of the present invention may be administered to a subject in an immunologically effective amount. The term “immunologically effective amount” refers to an amount sufficient to exhibit the effect of preventing or treating SARS-CoV-2 infection, and an amount that does not cause side effects or serious or excessive immune responses. The exact dose of the antigenic composition or vaccine may vary depending on the specific immunogen to be administered, and may be easily determined by those skilled in the art depending on factors well known in the medical field, including the age, body weight, health and sex of a subject to be prevented or treated, the drug sensitivity of the subject, the route of administration, and the mode of administration. The antigenic composition or vaccine may be administered once or several times.

The vaccine of the present invention is administered in a pharmaceutically effective amount. The term “pharmaceutically effective amount” refers to an amount sufficient to exhibit a vaccination effect, and an amount that does not cause side effects or serious or excessive immune responses. The exact dose of the vaccine may vary depending on the antigen to be administered, and may be easily determined by those skilled in the art depending on factors well known in the medical field, including the age, body weight, health and sex of a subject, the drug sensitivity of the subject, the route of administration, and the mode of administration. The vaccine may be administered once or several times.

According to a specific embodiment of the present invention, the transformant expresses SARS-CoV-2 recombinant protein.

In the present specification, the term “SARS-CoV-2” refers to a positive-sense, single-stranded RNA coronavirus based on DNA sequencing. The virus is contagious to humans and is the cause of COVID-19.

According to a specific embodiment of the present invention, the composition is administered intramuscularly, administered intranasally, or inhaled intranasally.

According to a specific embodiment of the present invention, the coronavirus is at least one selected from the group consisting of human coronavirus 229E (HCoV-229E), human coronavirus OC43 (HCoV-OC43), human coronavirus HKU1 (HCoV-HKU1), human coronavirus NL63 (HCoV-NL63), severe acute respiratory syndrome coronavirus (SARS-CoV), severe acute respiratory syndrome virus-2 (SARS-CoV-2), Middle East respiratory syndrome coronavirus (MERS-CoV), porcine epidemic diarrhea virus (PEDV), transmissible gastroenteritis virus (TGEV), porcine hemagglutinating encephalomyelitis virus (PHEV), bovine coronavirus (BCoV), equine coronavirus (EqCoV), murine coronavirus (MuCoV), canine coronavirus (CCoV), feline coronavirus (FCoV), Miniopterus bat coronavirus-1, Miniopterus bat coronavirus HKU8, Rhinolophus bat coronavirus HKU2, Scotophilus bat coronavirus 512, Tylonycteris bat coronavirus HKU4, Pipistrellus bat coronavirus HKU5, Rousettus bat coronavirus HKU9, avian coronavirus, Beluga whale coronavirus SW1, Bulbul coronavirus HKU11, Thrush coronavirus HKU12, and Munia coronavirus HKU13.

According to another aspect of the present invention, the present invention provides a coronavirus disease (COVID-19) prime-boost vaccine composition comprising, as an active ingredient, a recombinant adenovirus obtained by transfecting an adenovirus with the recombinant expression vector and culturing the transfected adenovirus.

In this specification, the term “prime boost” refers to a vaccination regimen. Prime generally refers to the first administration of a vaccine against a specific infection, which allows our body to build immunity against that disease. Boost refers to administering another dose against the same infection. Our body's immune cells basically remember previously received vaccines and respond much faster and stronger to subsequent vaccinations, building immunity to a level that protects our bodies.

According to another aspect of the present invention, the present invention provides a pharmaceutical composition for preventing or treating coronavirus disease (COVID-19), comprising, as an active ingredient, a recombinant adenovirus obtained by transfecting an adenovirus with the recombinant expression vector and culturing the transfected adenovirus.

In the present invention, since the prevention or treatment of coronavirus infection using the recombinant adenovirus has already been described in detail, the description thereof will be omitted to avoid excessive overlapping.

In the present specification, the term “pharmaceutical composition may be in the form of capsules, tablets, granules, or injections, ointments, powders, or beverages, and the pharmaceutical composition may be for administration to humans.

For use, the pharmaceutical composition of the present invention may be formulated in the form of oral preparations, including powders, granules, capsules, tablets or aqueous suspensions, inhalation formulations such as sprays, skin external preparations, suppositories, and sterile injectable solutions, according to respective conventional methods, without being limited thereto. The pharmaceutical composition of the present invention may comprise pharmaceutically acceptable carriers. As the pharmaceutically acceptable carriers, a binder, a lubricant, a disintegrant, an excipient, a solubilizer, a dispersant, a stabilizer, a suspending agent, a colorant, a flavoring agent, and the like may be used for oral administration; a buffer, a preservative, a pain-relieving agent, a solubilizer, an isotonic agent, a stabilizer, and the like may be used for injection; and a base, an excipient, a lubricant, a preservative, and the like may be used for topical administration. In addition, the pharmaceutical composition of the present invention may be prepared in various dosage forms by being mixed with the pharmaceutically acceptable carriers as described above. For example, for oral administration, the pharmaceutical composition may be formulated in the form of tablets, troches, capsules, elixirs, suspensions, syrups, wafers, or the like. For injection, the pharmaceutical composition may be formulated in the form of unit dosage ampoules or in multiple-dosage forms. In addition, the pharmaceutical composition may be formulated into solutions, suspensions, tablets, capsules, sustained-release preparations, or the like.

Meanwhile, examples of carriers, excipients and diluents suitable for formulation include lactose, dextrose, sucrose, sorbitol, mannitol, xylitol, erythritol, maltitol, starch, gum acacia, alginate, gelatin, calcium phosphate, calcium silicate, cellulose, methyl cellulose, microcrystalline cellulose, polyvinyl pyrrolidone, water, methylhydroxy benzoate, propylhydroxy benzoate, talc, magnesium stearate, and mineral oil. In addition, the pharmaceutical composition of the present invention may further contain a filler, an anticoagulant, a lubricant, a wetting agent, a fragrance, an emulsifier, a preservative, or the like.

The pharmaceutical composition of the present invention may vary depending on various factors, including the activity of a particular compound used, the patient's age, body weight, general health, sex and diet, administration time, the route of administration, excretion rate, drug combination, and the severity of a particular disease to be prevented or treated. Although the dose of the pharmaceutical composition varies depending on the patient's condition, body weight, the severity of the disease, the form of drug, the route of administration, and the duration of administration, it may be appropriately selected by a person skilled in the art. The pharmaceutical composition may be administered at a dose of 0.0001 to 50 mg/kg/day or 0.001 to 50 mg/kg/day. The pharmaceutical composition may be administered once or several times a day. The dose does not limit the scope of the present invention in any way. The pharmaceutical composition according to the present invention may be formulated as pills, sugar-coated tablets, capsules, liquids, gels, syrups, slurries, or suspensions.

According to another aspect of the present invention, the present invention provides a method for preventing or treating coronavirus disease (COVID-19), comprising a step of administering a recombinant adenovirus obtained by transfecting an adenovirus with the recombinant expression vector according to claim 1 and culturing the transfected adenovirus.

According to another aspect of the present invention, the present invention provides the use of a recombinant adenovirus, obtained by transfecting an adenovirus with the recombinant expression vector according to claim 1 and culturing the transfected adenovirus, as an active ingredient for preventing or treating coronavirus disease (COVID-19).

### Advantageous Effects

The features and advantages of the present invention are summarized as follows:
(a) The present invention provides a vaccine composition for preventing or treating coronavirus disease (COVID-19) comprising a recombinant adenovirus as an active ingredient.
(b) The present invention enhances immune responses against severe acute respiratory syndrome coronavirus 2 (SARS-CoV-2), which is a severe pandemic that has resulted in millions of deaths worldwide, through the recombinant adenovirus, and thus may be useful as a prophylactic vaccine composition that provides fundamental and efficient protection against SARS-CoV-2.

### BRIEF DESCRIPTION OF DRAWINGS

FIG. 1A is a schematic diagram showing a vaccination schedule for generating immunogenicity according to one embodiment of the present invention.

FIG. 1B shows results indicating the responses of T cells that secrete IFN-γ in the mediastinal lymph node (mLN) by antigen-specific responses after vaccination with a recombinant adenovirus vector via different vaccination routes (IM-IM or IM-IN), according to one embodiment of the present invention.

FIG. 1C shows results indicating the S RBD-specific antibody titers in blood and bronchoalveolar lavage fluid after vaccination with a recombinant adenovirus vector via different vaccination routes (IM-IM or IM-IN), according to one embodiment of the present invention.

FIG. 1D shows results indicating changes in the number of cells in the mLN and lung after vaccination with a recombinant adenovirus vector via different vaccination routes (IM-IM or IM-IN), according to one embodiment of the present invention.

FIG. 1E shows results indicating changes in the number of CD4 and CD8 resident memory T cells in the lung after vaccination with a recombinant adenovirus vector via different vaccination routes (IM-IM or IM-IN), according to one embodiment of the present invention.

FIG. 2A shows a vaccination schedule for constructing a recombinant adenovirus vector carrying the spike(S) as an antigen, which contains a D614G mutation and also contains 2P substitutions to maintain the trimeric prefusion conformation, according to one experimental example of the present invention.

FIG. 2B shows results indicating the responses of T cells that secrete IFN-γ in the spleen by antigen-specific responses, according to one experimental example of the present invention.

FIG. 2C shows results indicating IgG antibody titers in serum, according to one experimental example of the present invention.

FIG. 2D shows results indicating IgA antibody titers in bronchoalveolar lavage fluid, according to one experimental example of the present invention.

FIG. 3A shows results indicating cell changes in the spleen after administration of an Ad5(SD_{614G} 2P) vaccine, according to one experimental example of the present invention.

FIG. 3B shows results indicating changes in lung cells after administration of an Ad5(SD_{614G} 2P) vaccine, according to one experimental example of the present invention.

FIG. 3C shows results indicating that germinal center B cells in the spleen further increase after administration of an Ad5(SD_{614G} 2P) vaccine, according to one experimental example of the present invention.

FIG. 4A shows flow cytometry results indicating changes in T cells and B cells in lung tissue after administration of an Ad5(SD_{614G} 2P) vaccine, according to one experimental example of the present invention.

FIG. 4B shows results indicating changes in B cells in lung tissue after administration of an Ad5(SD_{614G} 2P) vaccine, according to one experimental example of the present invention.

FIG. 4C shows results indicating changes in T cells in lung tissue after administration of an Ad5(SD_{614G} 2P) vaccine, according to one experimental example of the present invention.

FIG. 4D shows results indicating changes in effector CD4T cells in lung tissue after administration of an Ad5(SD_{614G} 2P) vaccine, according to one experimental example of the present invention.

FIG. 4E shows results indicating changes in effector CD8T cells in lung tissue after administration of an Ad5(SD_{614G} 2P) vaccine, according to one experimental example of the present invention.

FIG. 5A shows flow cytometry results indicating changes in resident memory T cells in lung tissue after administration of an Ad5(SD_{614G} 2P) vaccine, according to one experimental example of the present invention.

FIG. 5B shows results indicating changes in CD4 resident memory T cells in lung tissue after administration of an Ad5(SD_{614G} 2P) vaccine, according to one experimental example of the present invention.

FIG. 5C shows results indicating changes in CD8 resident memory T cells in lung tissue after administration of an Ad5(SD_{614G} 2P) vaccine, according to one experimental example of the present invention.

FIG. 6 shows a vaccination schedule for constructing a recombinant adenovirus vector carrying an S-antigen, which contains a D614G mutation, together with human CXCL9, according to one experimental example of the present invention.

FIG. 7A shows results indicating the responses of T cells that secrete IFN-γ in the spleen by antigen-specific responses after vaccination with a vaccine co-expressing S-antigen and CXCL9, according to one experimental example of the present invention.

FIG. 7B shows results showing IgA antibody titers in bronchoalveolar lavage fluid after vaccination with a vaccine co-expressing S-antigen and CXCL9, according to one experimental example of the present invention.

FIG. 7C shows results showing IgG antibody titers in serum after vaccination with a vaccine co-expressing S-antigen and CXCL9, according to one experimental example of the present invention.

FIG. 8A shows results indicating cell changes in the spleen after vaccination with a vaccine co-expressing S-antigen and CXCL9, according to one experimental example of the present invention.

FIG. 8B shows results indicating changes in lung cells after vaccination with a vaccine co-expressing S-antigen and CXCL9, according to one experimental example of the present invention.

FIG. 8C shows results indicating changes in germinal center B cells in the spleen after vaccination with a vaccine co-expressing S-antigen and CXCL9, according to one experimental example of the present invention.

FIG. 9 shows results indicating changes in effector CD4T cells and resident memory CD4T cells in lung tissue after vaccination with a vaccine co-expressing S-antigen and CXCL9, according to one experimental example of the present invention.

FIG. 10 shows results indicating changes in effector CD8T cells and resident memory CD8T cells in lung tissue after vaccination with a vaccine co-expressing S-antigen and CXCL9, according to one experimental example of the present invention.

FIG. 11 shows a vaccination schedule for constructing a recombinant adenovirus vector carrying an S-antigen, which contains a D614G mutation, together with human IL-7, according to one experimental example of the present invention.

FIG. 12A shows results indicating the responses of T cells that secrete IFN-γ in the spleen by antigen-specific responses after vaccination with a vaccine co-expressing S-antigen and IL-7, according to one experimental example of the present invention.

FIG. 12B shows results showing IgA antibody titers in bronchoalveolar lavage fluid after vaccination with a vaccine co-expressing S-antigen and IL-7, according to one experimental example of the present invention.

FIG. 12C shows results showing IgG antibody titers in serum after vaccination with a vaccine co-expressing S-antigen and IL-7, according to one experimental example of the present invention.

FIG. 13A shows results indicating cell changes in the spleen after vaccination with a vaccine co-expressing S-antigen and IL-7, according to one experimental example of the present invention.

FIG. 13B shows results indicating changes in lung cells after vaccination with a vaccine co-expressing S-antigen and IL-7, according to one experimental example of the present invention.

FIG. 13C shows results indicating changes in germinal center B cells in the spleen after vaccination with a vaccine co-expressing S-antigen and IL-7, according to one experimental example of the present invention.

FIG. 14 shows results indicating changes in effector CD4T cells and resident memory CD4T cells in lung tissue after vaccination with a vaccine co-expressing S-antigen and IL-7, according to one experimental example of the present invention.

FIG. 15 shows results indicating changes in effector CD8T cells and resident memory CD8T cells in lung tissue after vaccination with a vaccine co-expressing S-antigen and IL-7, according to one experimental example of the present invention.

FIG. 16 shows a vaccination schedule for constructing a recombinant adenovirus vector vaccine co-expressing antigen SD_{614G} 2P and human CXCL9, according to one experimental example of the present invention.

FIG. 17 shows the results of comparing antigen-specific responses in serum after vaccination with Ad5(SD_{614G} 2P) and Ad5(S_{D614G} 2P-CXCL9) recombinant adenovirus vector vaccines, according to one experimental example of the present invention.

FIG. 18 shows a vaccination schedule for confirming whether vaccination with a constructed recombinant adenovirus vector vaccine co-expressing antigen S_{D614G} 2P and human CXCL9 can protect the host upon SARS-CoV-2 infection, according to one experimental example of the present invention.

FIG. 19A shows the results of comparing antibody titers in serum after vaccination with Ad5(SD_{614G} 2P) and Ad5(S_{D614G} 2P-CXCL9) recombinant adenovirus vector vaccines, according to one experimental example of the present invention.

FIG. 19B shows the results of comparing body weight changes and survival rates after infection with live SARS-CoV-2 virus following vaccination with Ad5(SD_{614G} 2P) and Ad5(S_{D614G} 2P-CXCL9) recombinant adenovirus vector vaccines, according to one experimental example of the present invention.

FIG. 20 is a schematic diagram showing an adenovirus vector-based recombinant vaccine according to one experimental example of the present invention.

### Best Mode

The present invention relates to the development of an effective vaccine against severe acute respiratory syndrome coronavirus 2 (SARS-CoV-2). In the present invention, a recombinant expression vector was developed using the coronavirus surface spike protein and an adjuvant, and it was found that the recombinant expression vector enhanced immune responses against the coronavirus when injected intranasally. Accordingly, the present invention can provide an important platform for the development of a fundamental and efficient vaccine composition against SARS-CoV-2.

### Mode for Invention

Hereinafter, the present invention will be described in more detail by way of examples. These examples are only for illustrating the present invention in more detail, and it will be apparent to those skilled in the art that the scope of the present invention according to the subject matter of the present invention is not limited by these examples.

### EXAMPLES

### [Example 1] Construction of Recombinant Adenovirus Vector Vaccines

The present inventors produced recombinant adenoviruses co-expressing the spike (S) protein derived from the SARS-CoV-2 virus, which causes COVID-19, and human CXCL9 or IL-7.

Specifically, vectors were designed by inserting a gene sequence (SEQ ID NO: 5) encoding a P2A sequence (SEQ ID NO: 4) between a gene sequence (SEQ ID NO: 1) encoding an S protein comprising an aspartic acid (D)-to-glycine (G) substitution at amino acid 614 of the S protein of SARS-CoV-2 virus, like the mutant virus which is currently dominant, and a gene sequence encoding human CXCL9 (SEQ ID NO: 2) or IL-7 (SEQ ID NO: 3), so that the S protein and CXCL9 or IL-7 would be expressed separately from each other in cells infected with the recombinant adenovirus. To this end, a P2A sequence-linked S gene PCR product was made using the primers of SEQ ID NOs: 6, 7 and 8, a P2A sequence-linked CXCL9 PCR product was made using the primers of SEQ ID NOs: 9, 10and 11, and a P2A sequence-linked IL-7 PCR product was made using the primers of SEQ ID NOs: 9, 12 and 13. Then, the S gene PCR product and the CXCL9 or IL-7 PCR product were subjected to overlap PCR, thus making an S_{D614G}-P2A-CXCL9 or S_{D614G}-P2A-IL-7 PCR product having KpnI and XbaI restriction enzyme cleavage sites introduced at both ends. After performing PCR, the amplified sequence was inserted into a pShuttle-CMV vector, thus constructing a recombinant pShuttle-CMV vector. Specifically, the pShuttle-CMV vector and the S_{D614G}-P2A-CXCL9 or S_{D614G}-P2A-IL-7 PCR product were treated with KpnI and XbaI restriction enzymes, and the PCR product was inserted into the vector using T4 DNA ligase, thereby constructing recombinant vectors, which were named pShuttle-CMV S_{D614G}-P2A-CXCL9 and pShuttle-CMV S_{D614G}-P2A-IL-7 vectors. Additionally, modifications were made to the S protein so that the S protein could be expressed while maintaining its pre-fusion form during vaccination. To this end, pShuttle-CMV S_{D614G} 2P and pShuttle-CMV S_{D614G} 2P-P2A-CXCL9 vectors expressing an S antigen having amino acid substitutions (K986P and V987P) were also constructed using the primers of SEQ ID NOs: 14 and 15.

Thereafter, the recombinant vectors were linearized by treatment with a PmeI restriction enzyme and co-transformed with a pAdEasy-1 vector into a BJ5183 strain, thereby constructing recombinant pAdEasy-1 vectors, which were named the pAdEasy-1 S_{D614G}-P2A-CXCL9 vector, the pAdEasy-1 S_{D614G}-P2A-IL-7 vector, the pAdEasy-1 S_{D614G} 2P vector, and the pAdEasy-1 S_{D614G} 2P-P2A-CXCL9 vector. These vectors were treated with a PacI restriction enzyme and transfected into Adeno X-293 cells to obtain recombinant adenoviruses. These recombinant adenoviruses were re-infected into Adeno X-293 cells several times, amplified, purified using an Adeno-X Maxi Purification Kit, and dialyzed in DPBS to obtain vaccine candidates Ad5(S_{D614G}-CXCL9), Ad5(S_{D614G}-IL-7), Ad5(SD_{614G} 2P), and Ad5(S_{D614G} 2P-CXCL9).

### [Example 2] Verification of Immunogenicity of Recombinant Adenovirus Vector Vaccines

A mouse model was used to verify the immunogenicity of the recombinant adenovirus vector vaccines constructed in Example 1. Specifically, 100 μL of the vaccine candidate was injected intramuscularly into the right hind thigh of BALB/c mice at a dose of 1.0×10¹⁰ VP/mouse at D0, and after 14 days, the mice were intranasally vaccinated with 20 μL of the adenovirus vector vaccine at a dose of 1.0×10¹⁰ VP/mouse. Blood was sampled from the orbital venous plexus of the mice at 7-day intervals starting from D0, and the titer of S RBD antigen-specific IgG antibody in the serum was analyzed by ELSIA. On day 28 after vaccination, the mice were euthanized, bronchoalveolar lavage fluid was collected, and the titers of S RBD antigen-specific IgG and IgA antibodies in the bronchoalveolar lavage fluid were analyzed. In addition, lung cells and spleen cells were isolated from the mice on day 28 after vaccination and subjected to flow cytometry. In addition, the spleen cells were subjected to ELISpot assay, and the number of T cells generating IFN-γ by antigen-specific responses when stimulated with the antigen peptide pool was measured.

### [Example 3] Optimization of Vaccination Route to Increase Immunogenicity

### 3-1. Prime-Pulling Strategy

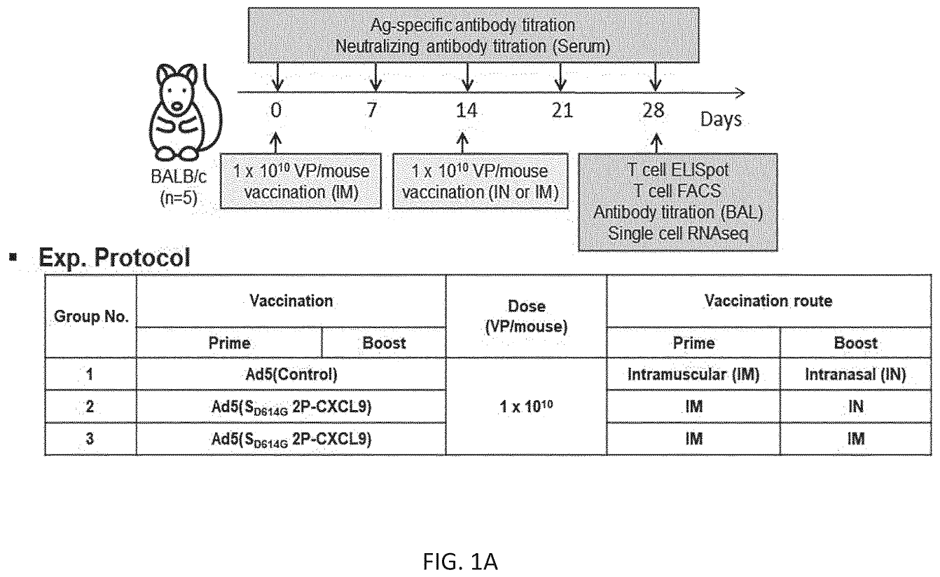

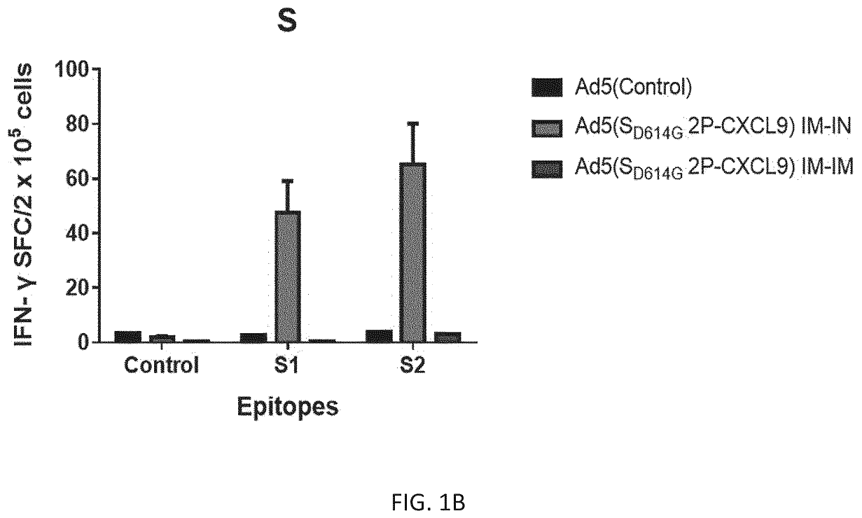

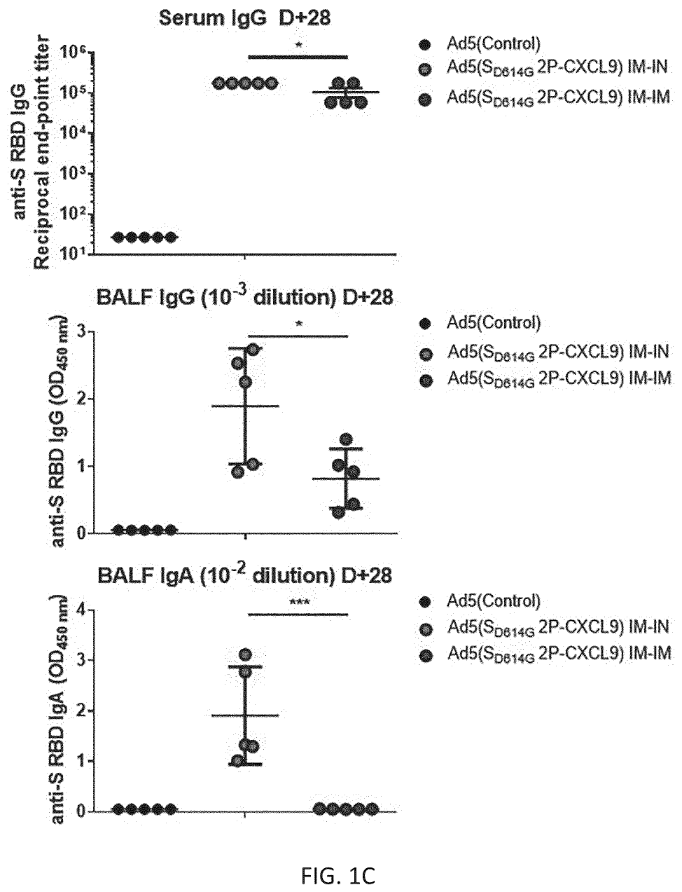

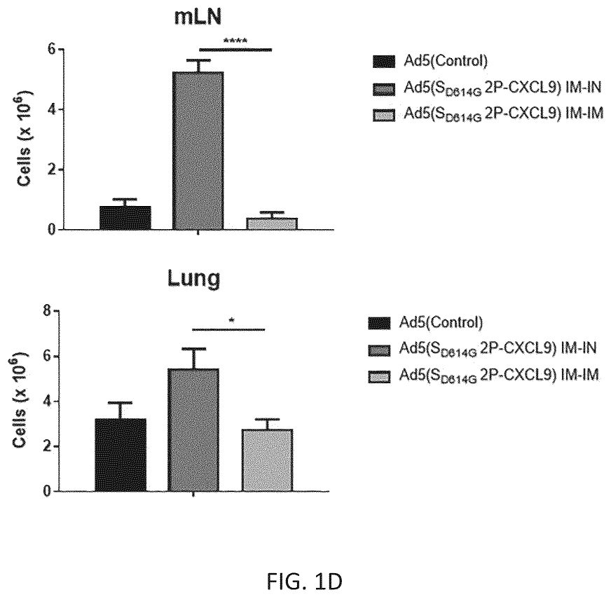

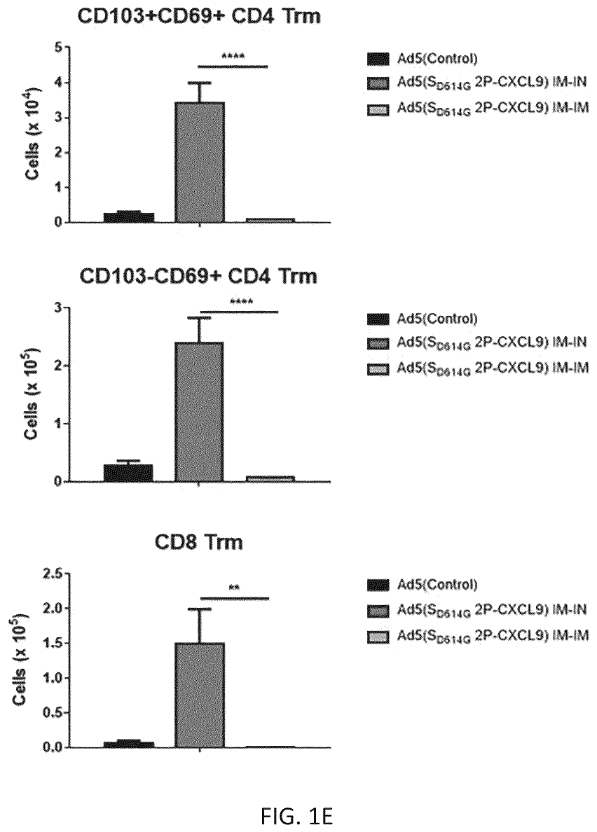

To optimize the vaccination route of the recombinant adenovirus vector vaccine, the recombinant adenovirus vector vaccine Ad5(S_{D614G} 2P-CXCL9) carrying the S_{D614G} 2P antigen and co-expressing CXCL9 was prime-injected intramuscularly into the right hind thigh of mice at a dose of 1.0×10¹⁰ VP/mouse, and after 2 weeks, at the time of boost, the mice were injected intramuscularly or vaccinated intranasally with the same dose. The immunogenicity between the IM-IM (intramuscular-intramuscular) and IM-IN (intramuscular-intranasal) vaccination routes was compared. As a result, it was found that, on 28 days after vaccination, the number of T cells that secrete IFN-γ in the mediastinal lymph node (mLN), which is the draining lymph node of intranasal vaccination, by antigen-specific responses, hardly increased in the IM-IM vaccination route, but significantly increased in the IM-IN vaccination route (FIG. 1B). The S antigen-specific antibody titers in serum and bronchoalveolar lavage fluid were higher when administered by the IM-IN route, and IgA antibodies, which are very important for mucosal immunity, were not identified in IM-IM vaccination, whereas the titers were higher when administered by the IM-IN route (FIG. 1C). In addition, it was found that both the numbers of mLN cells and lung cells increased when vaccinated by the IM-IN route (FIG. 1D), and the number of CD4 and CD8 resident memory T cells in the lung also increases only when administered by the IM-IN route (FIG. 1E). This suggests that intramuscular injection for the first vaccination followed by intranasal vaccination for the second vaccination is the method that can induce mucosal immune responses and maximize the efficiency of the vaccine.

### [Example 4] Selection of Antigen That Efficiently Increases Immune Responses

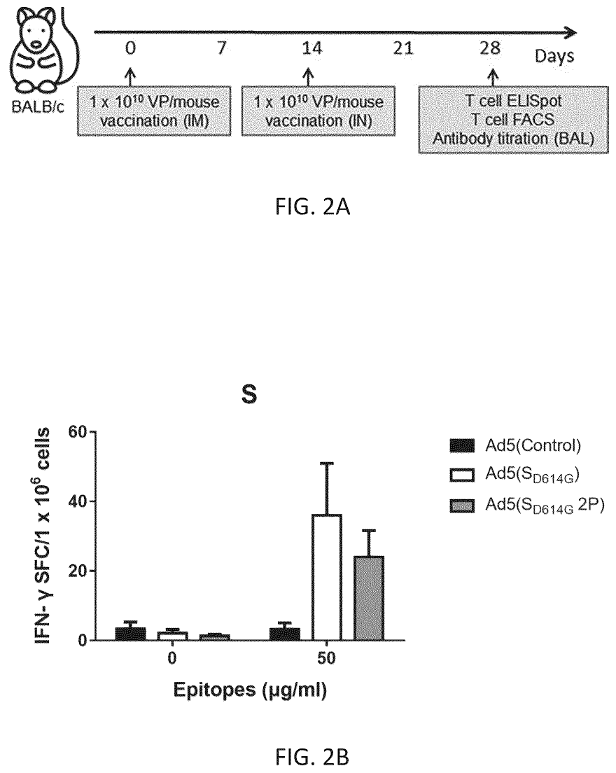

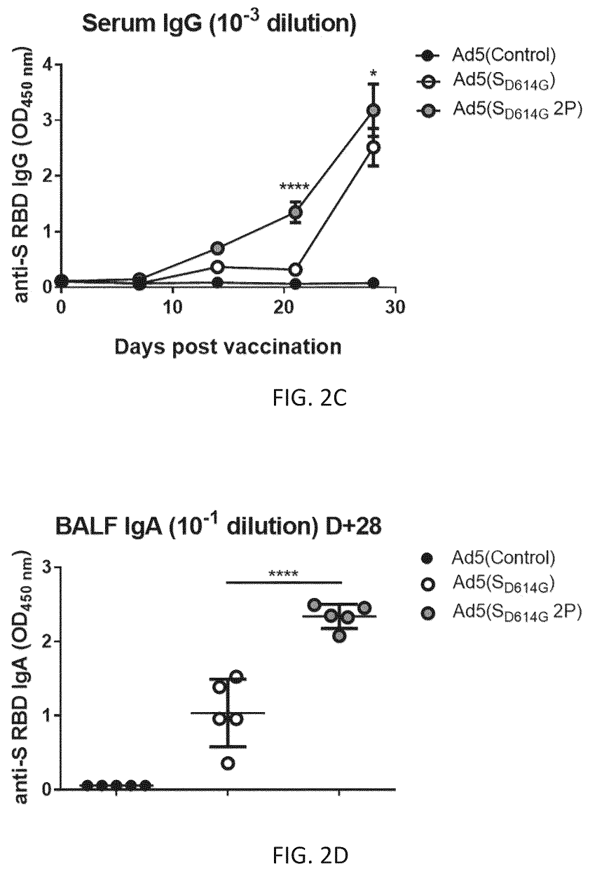

After constructing a recombinant adenovirus vector carrying an S antigen, which contains the D614G mutation of the currently prevailing spike(S) protein and also contains 2P substitutions to maintain the trimeric prefusion conformation, immunogenicity evaluation of the recombinant adenovirus vector was performed. As a result, it was found that there was no significant difference in the T cells secreting IFN-γ in the spleen by antigen-specific responses (FIG. 2B), but the experimental group vaccinated with the S 2P stabilized in its prefusion form had more S RBD protein-specific antibodies in the serum and bronchoalveolar lavage fluid than the control group (FIG. 2).

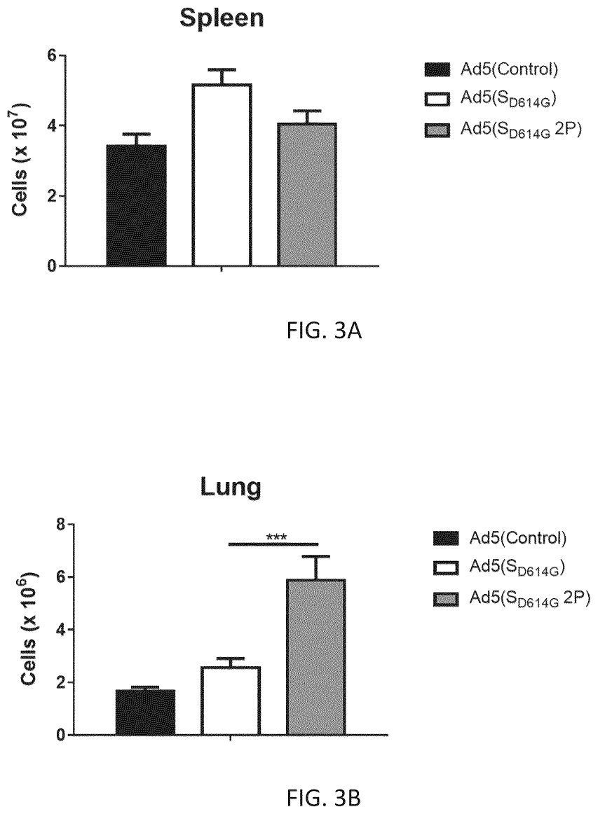

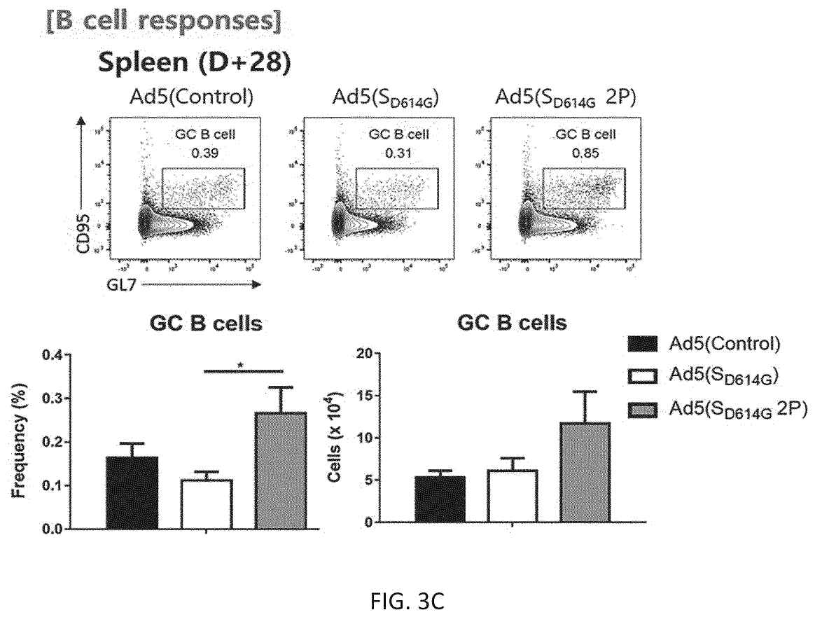

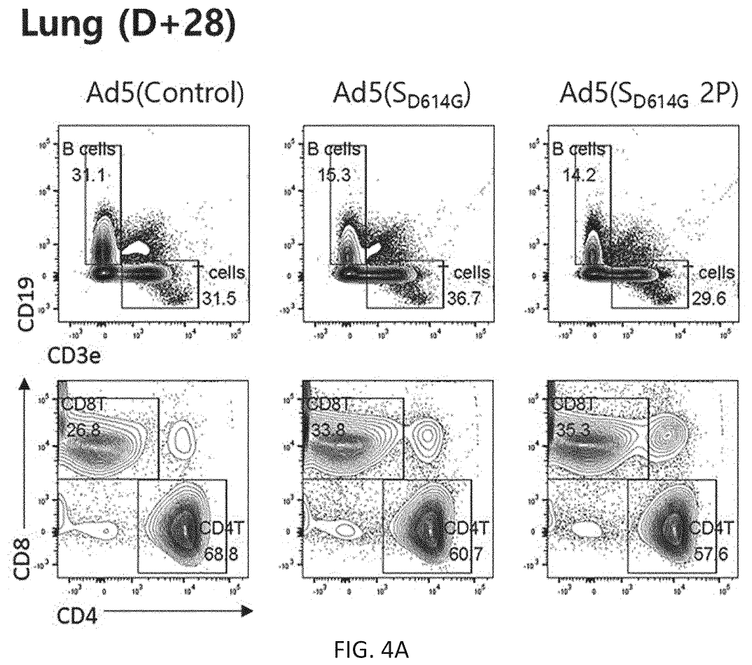

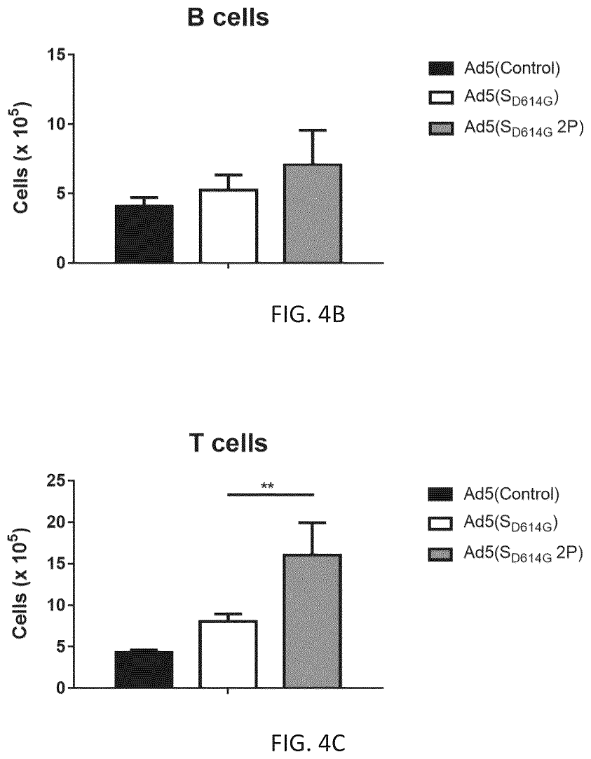

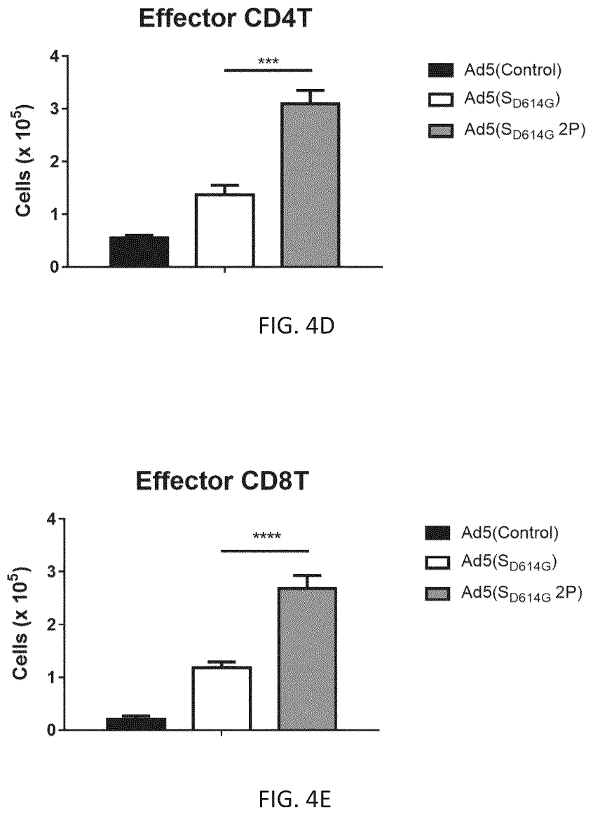

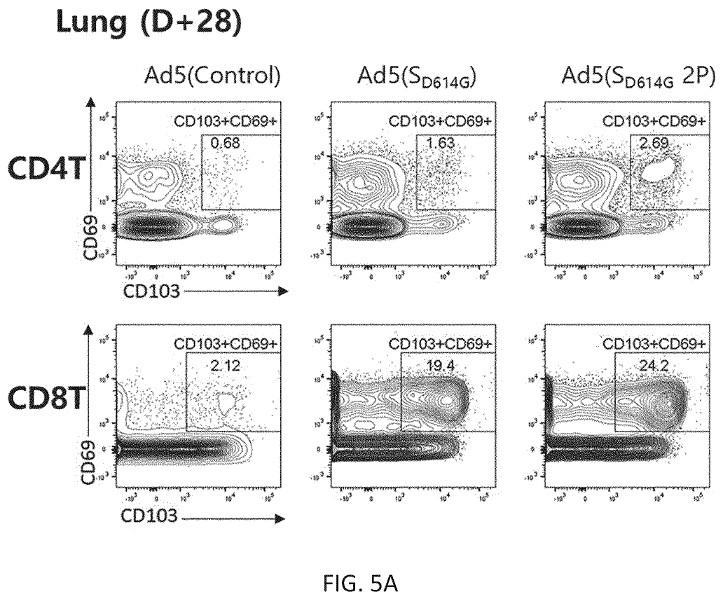

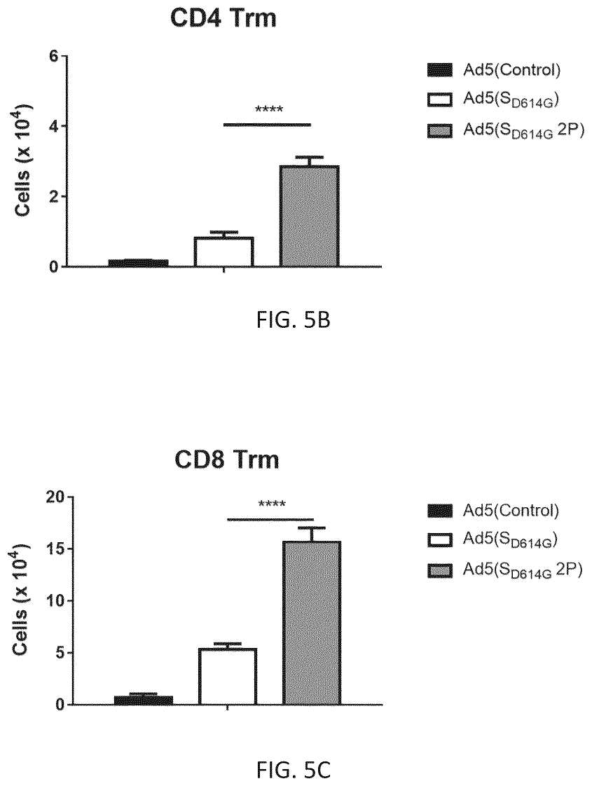

It was found that, in the experimental group administered the Ad5(SD_{614G} 2P) vaccine, lung cells increased compared to those in the control group, and germinal center B cells in the spleen further increased (FIG. 3). In addition, it was found that T cells in the lung tissue generally increased (FIG. 4), and the experimental group vaccinated with the Ad5(SD_{614G} 2P) vaccine had increased resident memory T cells in the lung tissue compared to the control group (FIG. 5).

### [Example 5] Selection of Adjuvant Vector That Efficiently Increases Immune Responses

### 5-1. Chemokine Ligand 9 (CXCL9)

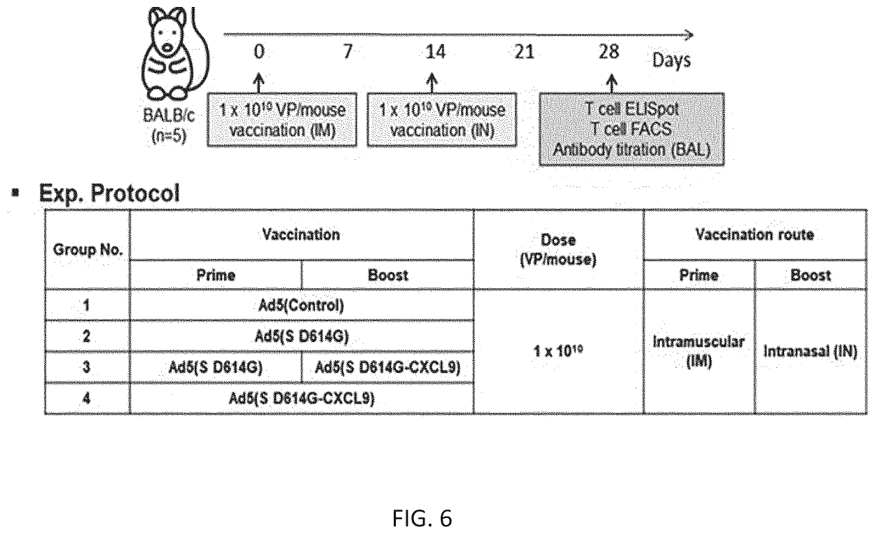

A recombinant adenovirus vector co-expressing the S antigen and human CXCL9 was constructed (FIG. 6), and immunogenicity thereof was evaluated to confirm the efficacy of the adjuvant.

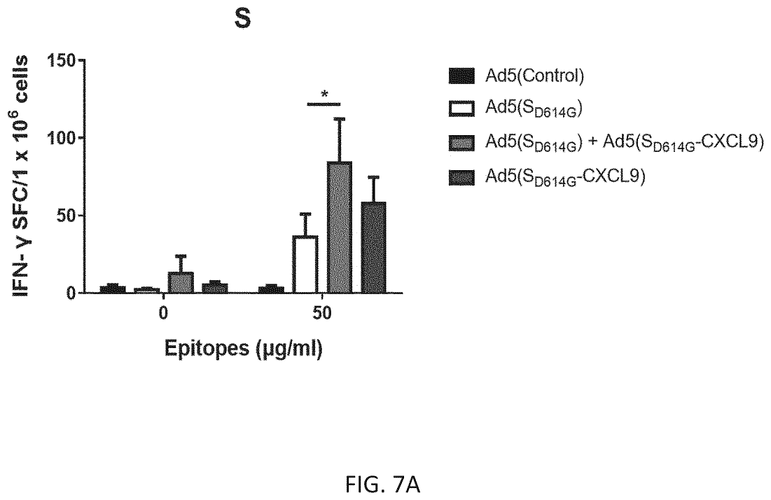

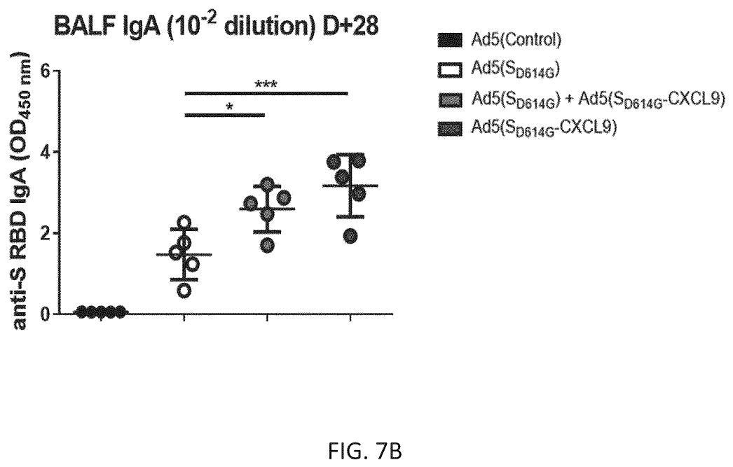

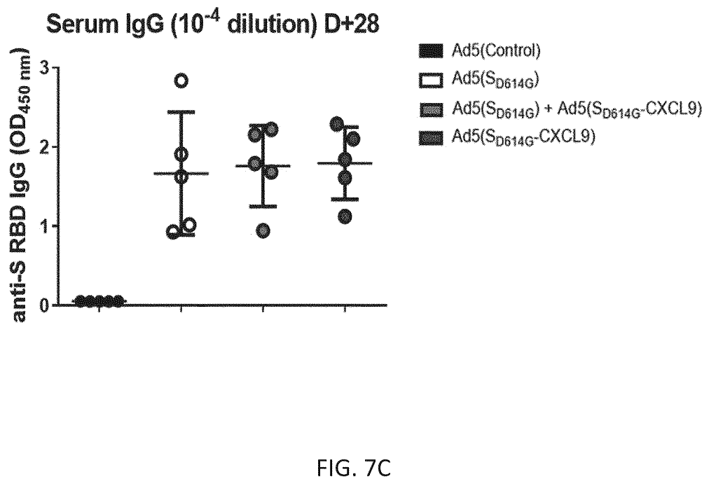

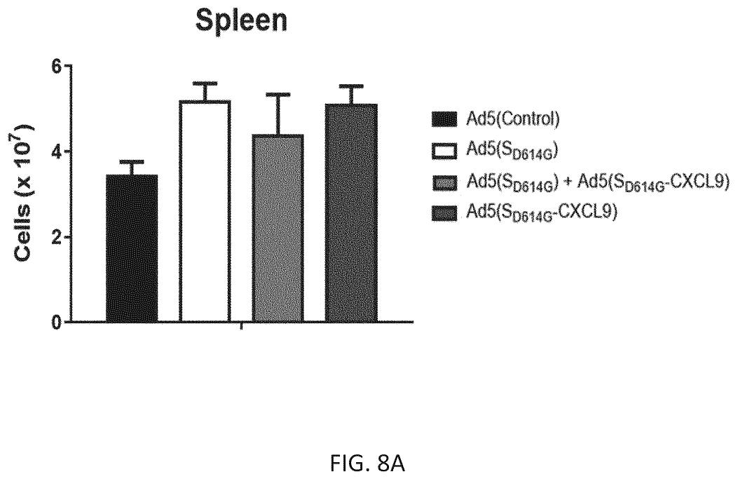

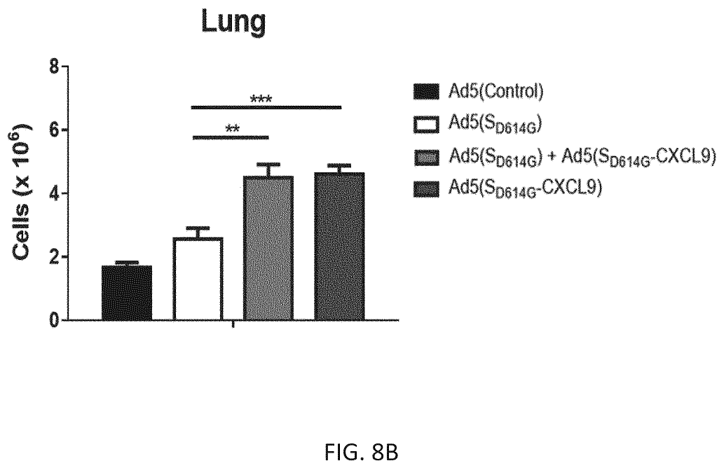

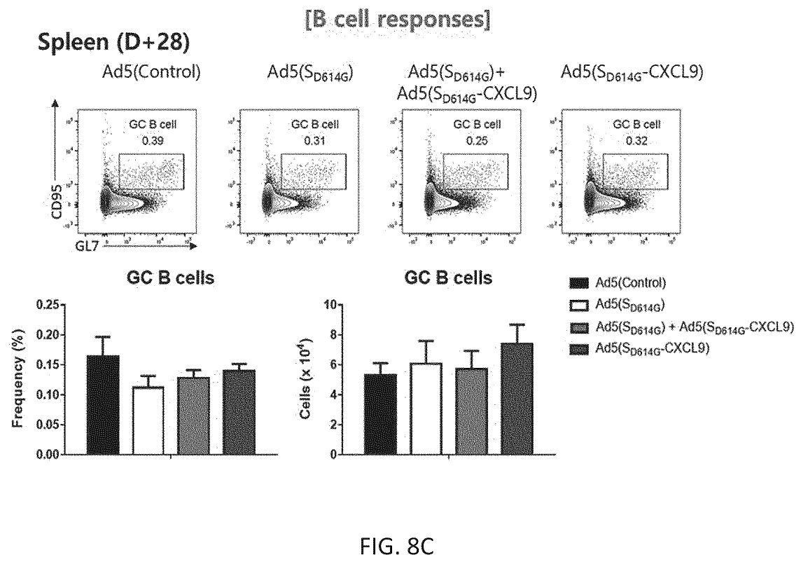

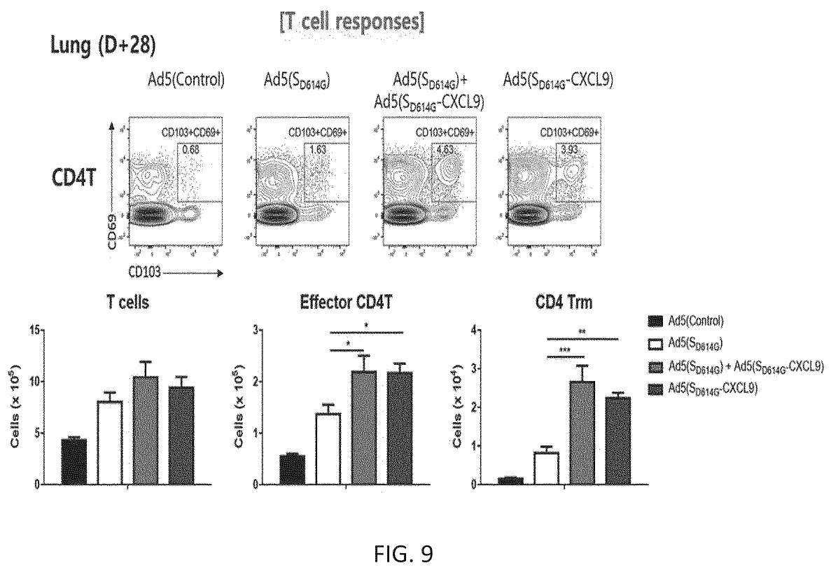

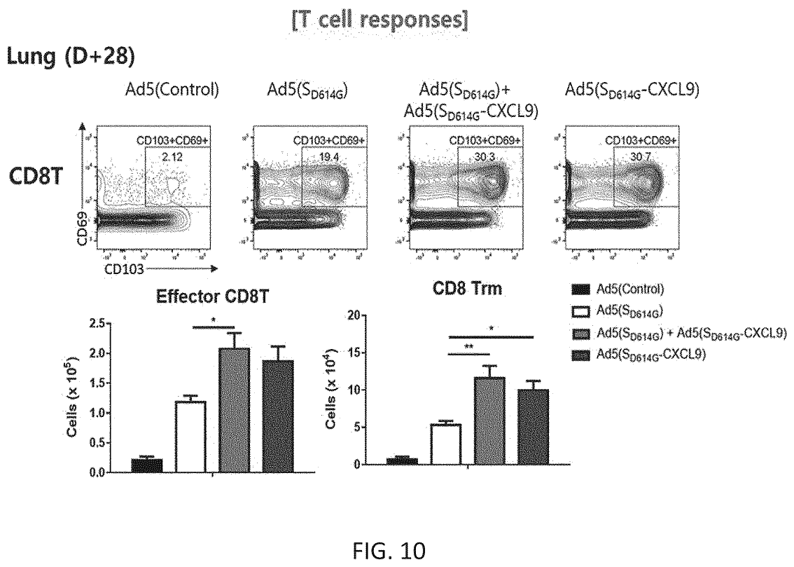

It was found that T cells that secrete IFN-γ in the spleen by antigen-specific responses generated more IFN-γ when vaccinated with the vaccine co-expressing the S antigen and CXCL9, and in particular, it could be confirmed that the method composed of the first vaccination with the vector expressing S alone and the second vaccination with the vector co-expressing S and CXCL9 was most efficient (FIG. 7). The titer of antigen-specific IgA in the bronchoalveolar lavage fluid (BALF) also increased when vaccinated with the vaccine co-expressing CXCL9, and there was no significant difference in antigen-specific IgG in the serum (FIG. 7). The number of lung cells tended to increase when vaccinated with the vaccine co-expressing the S antigen and CXCL9, and there was no difference in germinal center B cells in the spleen between the vaccinated groups (FIG. 8). It was confirmed that effector CD4T cells and resident memory CD4T cells in the lung tissue clearly tended to increase when vaccinated with the vaccine co-expressing the S antigen and CXCL9 (FIG. 9), and that effector CD8T cells and resident memory CD8T cells in the lung tissue tended to increase when vaccinated with the vaccine co-expressing the S antigen and CXCL9 (FIG. 10).

### 5-2. Interleukin 7 (IL-7)

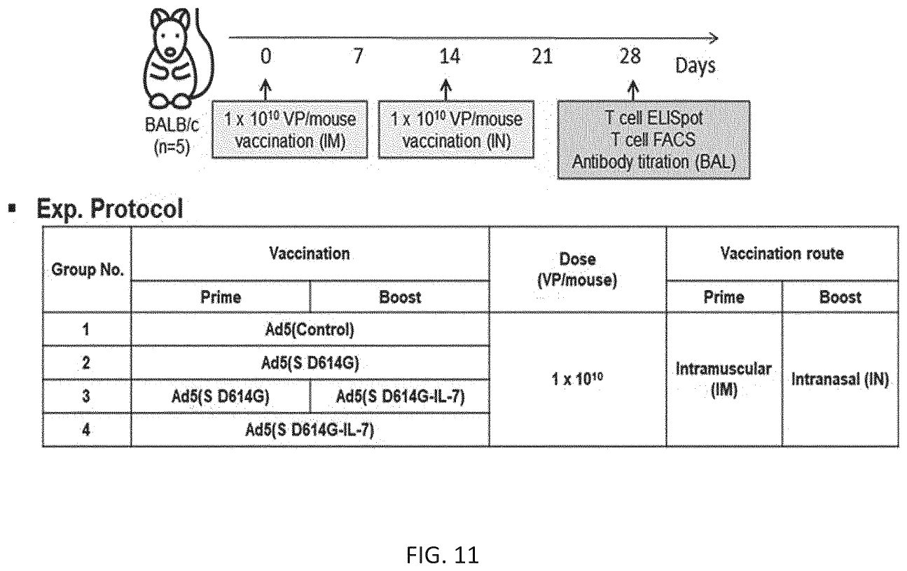

A recombinant adenovirus vector co-expressing the S antigen and human IL-7 was constructed (FIG. 11), and immunogenicity thereof was evaluated to confirm the efficiency of the adjuvant.

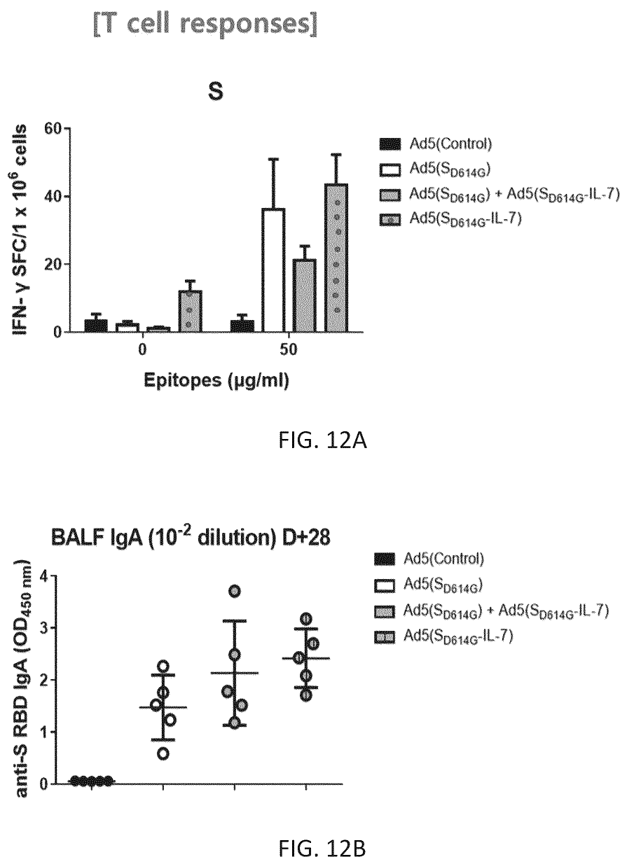

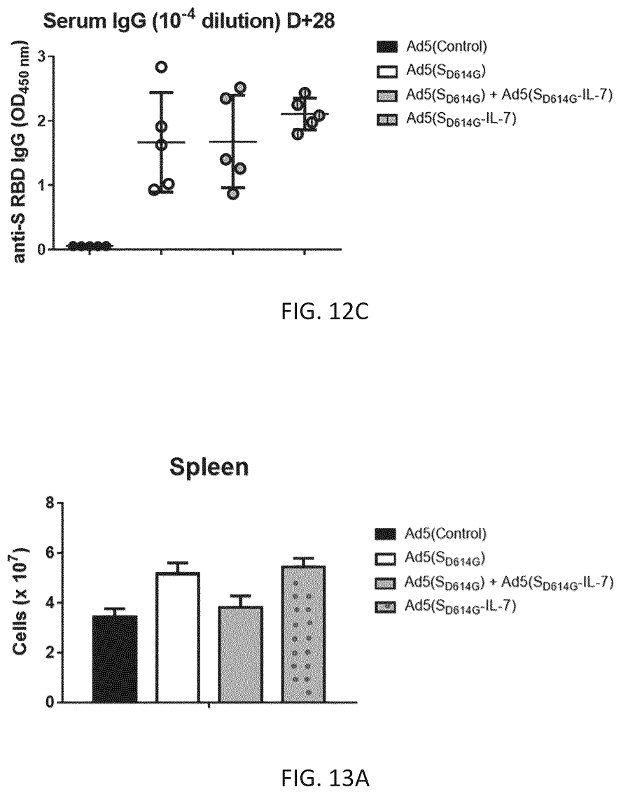

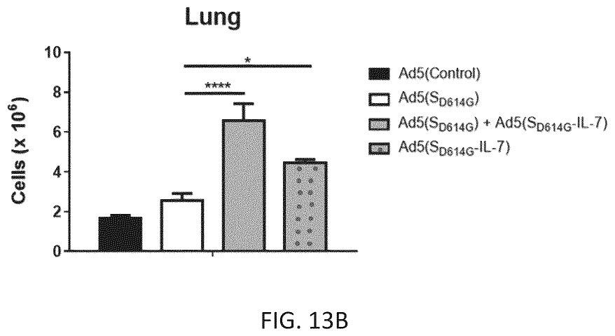

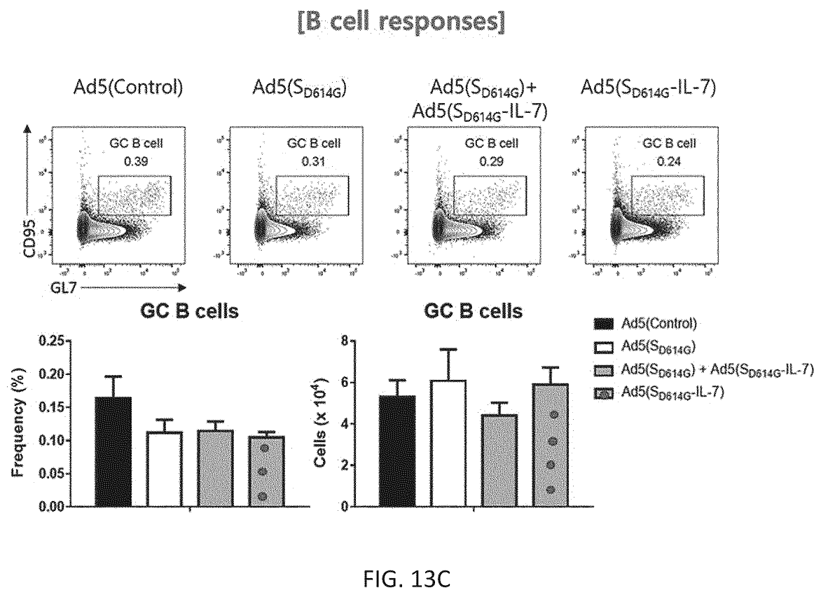

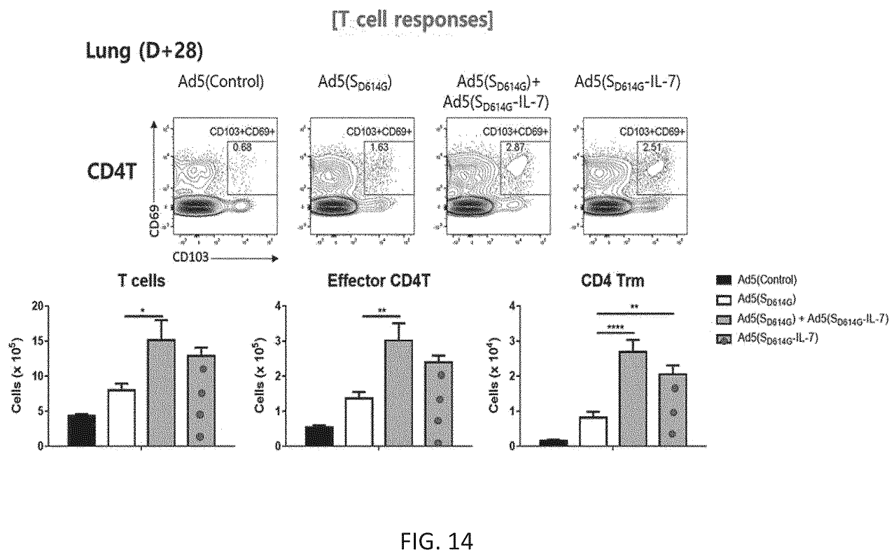

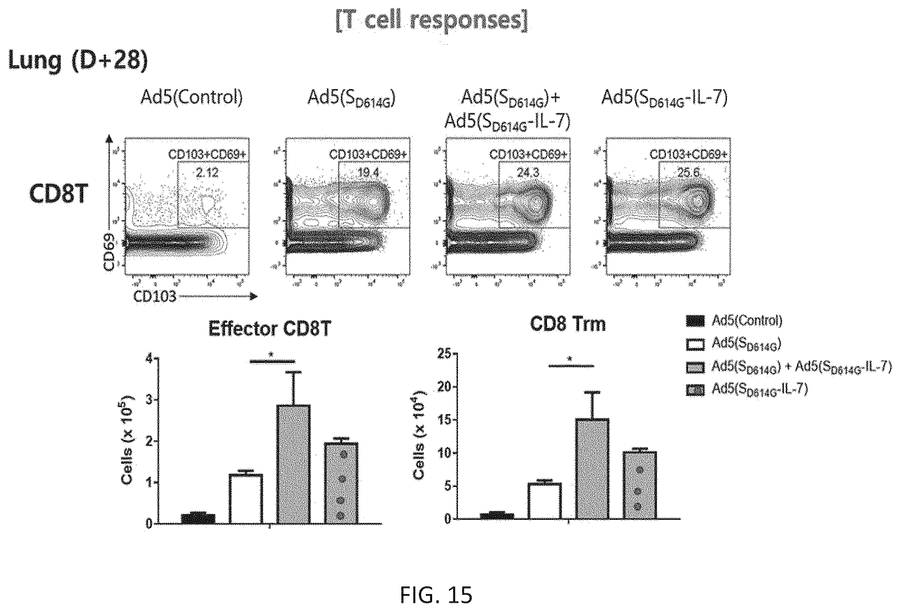

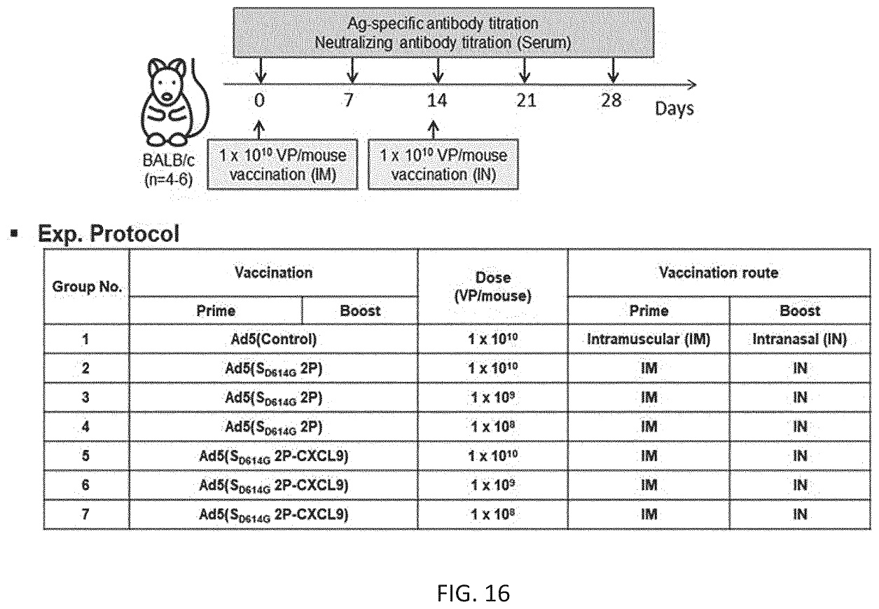

When vaccinated with the vector co-expressing the S antigen and IL-7, there was no significant difference in T cells that respond specifically to the antigen and in antibody formation (FIG. 12), the number of lung cells tended to increase when vaccinated with the vaccine co-expressing the S antigen and IL-7, and there was no difference in germinal center B cells in the spleen between the vaccinated groups (FIG. 13). It was confirmed that resident memory CD4T cells in the lung tissue tended to increase when vaccinated with the vaccine co-expressing the S antigen and IL-7, and the method composed of the first vaccination with the vector expressing S alone and the second vaccination with the vector co-expressing S and IL-7 was most efficient (FIG. 14). It was confirmed that resident memory CD8T cells in the lung tissue tended to increase when vaccinated with the vaccine co-expressing the S antigen and IL-7, and the method composed of the first vaccination with the vector expressing S alone and the second vaccination with the vector co-expressing S and IL-7 was most efficient (FIG. 15).

### Example 6

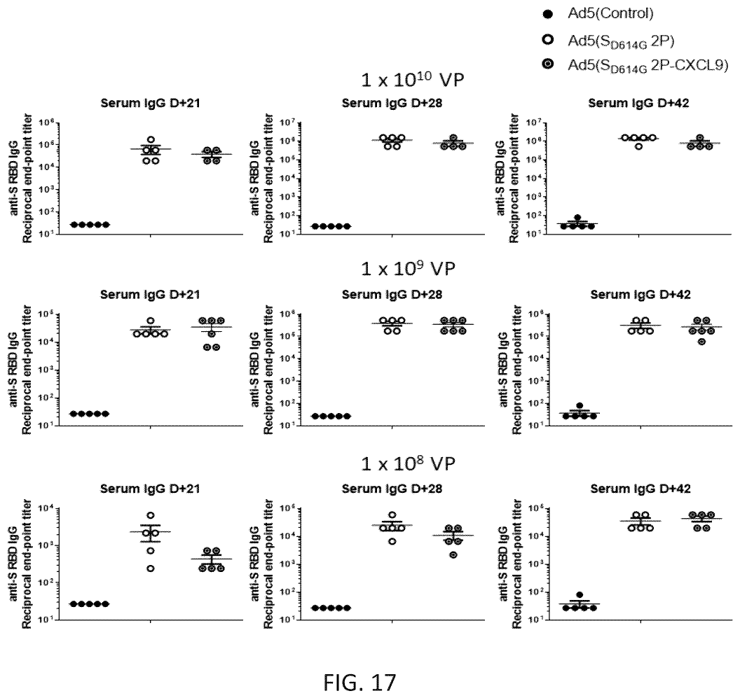

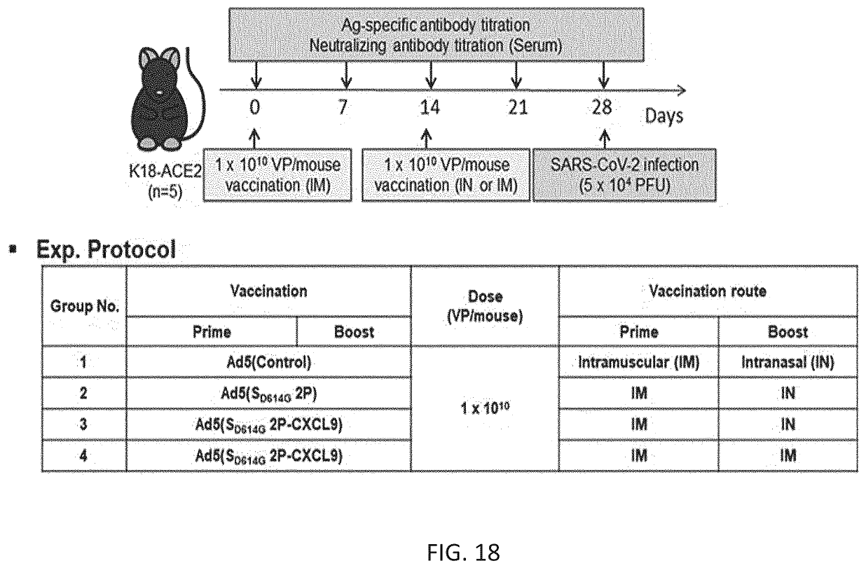

A recombinant adenovirus vector vaccine co-expressing the antigen S_{D614G} 2P and the adjuvant human CXCL9 in one virus was constructed and the difference in immunogenicity thereof from a recombinant adenovirus vector vaccine expressing only the antigen S_{D614G} 2P was examined. Specifically, mice were vaccinated with each of the Ad5(SD_{614G} 2P) and Ad5(S_{D614G} 2P-CXCL9) recombinant adenovirus vector vaccine at doses of 1.0×10⁸, 1.0×10⁹, and 1.0×10¹⁰ VP/mouse, and on 14 days after the first IM vaccination, the second IN vaccination was performed, and antigen-specific antibody titers in serum were compared at 1-2-week intervals. As a result, it was confirmed that both Ad5(SD_{614G} 2P) and Ad5(S_{D614G} 2P-CXCL9) vaccines well produced antibodies that bind specifically to the S antigen, without significant differences (FIG. 17).

### Example 7

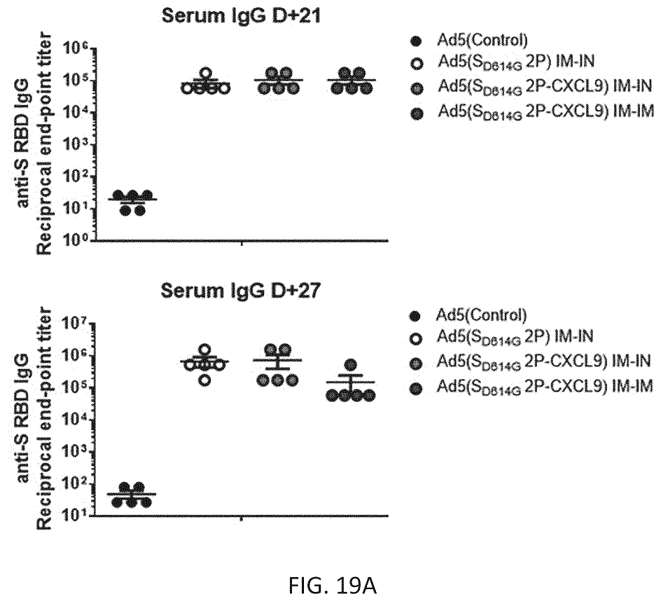

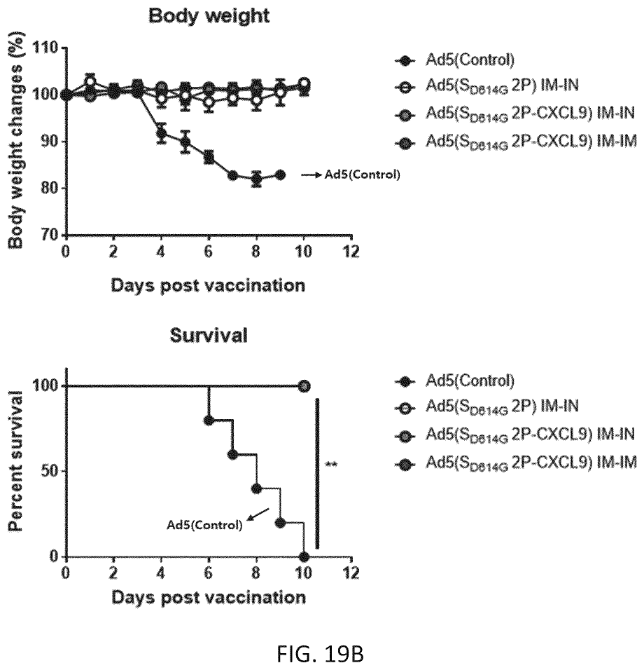

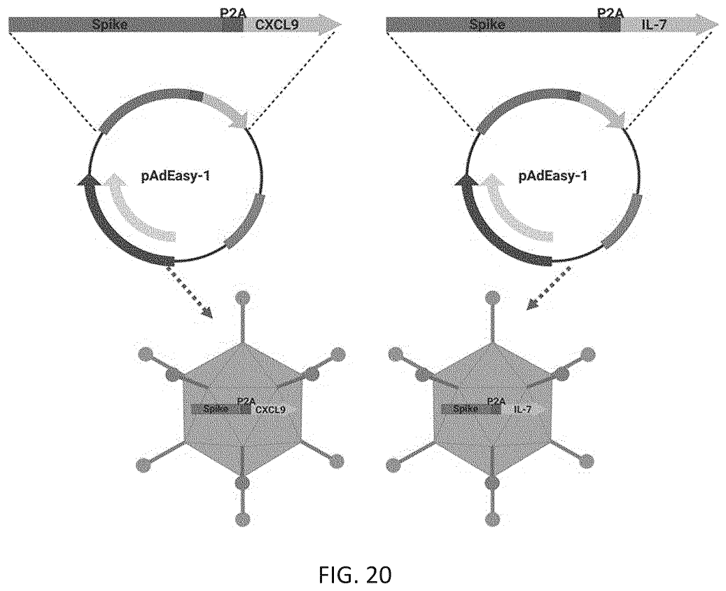

A recombinant adenovirus vector vaccine co-expressing the antigen S_{D614G} 2P and the adjuvant human CXCL9 in one virus was constructed, and whether vaccination with the vaccine can protect the host upon SARS-CoV-2 infection was examined. To this end, K18-ACE2 mice highly sensitive to SARS-CoV-2 were used. Specifically, in the first vaccination, the vaccine was administered at a dose of 1.0×10¹⁰ VP/mouse by the IM vaccination route, and in the second vaccination after 14 days, the same dose of the vaccine was administered by the IN vaccination route or the IM vaccination route. Then, on D +28, 14 days after the second vaccination, the mice were infected intranasally with live SARS-CoV-2 virus at a titer of 5×10⁴ PFU, and weight change and survival rate of the mice were checked for 10 days. As a result, the titer of S antigen-specific antibodies in the serum at 3 and 4 weeks after the start of vaccination did not significantly differ between the Ad5(SD_{614G} 2P) and Ad5(S_{D614G} 2P-CXCL9) recombinant adenovirus vector vaccines administered by the IM-IN route, and the Ad5(S_{D614G} 2P-CXCL9) vaccine showed no difference in the serum antibody titer even when administered by different routes (the IM-IN route and the IM-IM route (FIG. 19A). In addition, when infected with the live SARS-CoV-2 virus, the mice of the Ad5 (control)-vaccinated group all died within 10 days, while the mice of the group vaccinated with each of Ad5(SD_{614G} 2P) and Ad5(S_{D614G} 2P-CXCL9) all lost little body weight and survived until day 10 (FIG. 19B).

Additionally, it was confirmed that the mice of the group vaccinated with the Ad5(S_{D614G} 2P-CXCL9) vaccine by the IM-IM route also lost little body weight and all survived, suggesting that, at a dose of 1×10¹⁰ VP/mouse, which is the current experimental condition, all of the vaccines can protect the host upon SARS-CoV-2 infection regardless of the vaccination route (FIG. 19B).

Although the present invention has been described in detail with reference to the specific features, it will be apparent to those skilled in the art that this description is only of a preferred embodiment thereof, and does not limit the scope of the present invention. Thus, the substantial scope of the present invention will be defined by the appended claims and equivalents thereto.

### Industrial Applicability

The present invention relates to the development of an effective vaccine against severe acute respiratory syndrome coronavirus 2 (SARS-CoV-2). Currently, research on a more fundamental and effective vaccine against coronavirus, which is responsible for a severe pandemic that has resulted in millions of deaths worldwide, is insignificant, and the need for a new vaccine is required. In the present invention, a recombinant expression vector has been developed using the coronavirus surface spike protein and an adjuvant, and it has been found that the recombinant expression vector enhances immune responses against the coronavirus when injected intranasally. Thus, it is expected that the recombinant expression vector will be used to develop prophylactic vaccine compositions that provide efficient protection against SARS-CoV-2.

Sequence Listing Free Text
SARS-COV-2 S D614G
[SEQ ID NO: 1]
ATGTTTGTGTTCCTGGTGCTGCTGCCACTGGTGTCCAGCCAGTGTGTGAA
CCTGACCACCAGGACCCAACTTCCTCCTGCCTACACCAACTCCTTCACCA
GGGGAGTCTACTACCCTGACAAGGTGTTCAGGTCCTCTGTGCTGCACAGC
ACCCAGGACCTGTTCCTGCCATTCTTCAGCAATGTGACCTGGTTCCATGC
CATCCATGTGTCTGGCACCAATGGCACCAAGAGGTTTGACAACCCTGTGC
TGCCATTCAATGATGGAGTCTACTTTGCCAGCACAGAGAAGAGCAACATC
ATCAGGGGCTGGATTTTTGGCACCACCCTGGACAGCAAGACCCAGTCCCT
GCTGATTGTGAACAATGCCACCAATGTGGTGATTAAGGTGTGTGAGTTCC
AGTTCTGTAATGACCCATTCCTGGGAGTCTACTACCACAAGAACAACAAG
TCCTGGATGGAGTCTGAGTTCAGGGTCTACTCCTCTGCCAACAACTGTAC
CTTTGAATATGTGAGCCAACCATTCCTGATGGACTTGGAGGGCAAGCAGG
GCAACTTCAAGAACCTGAGGGAGTTTGTGTTCAAGAACATTGATGGCTAC
TTCAAGATTTACAGCAAACACACACCAATCAACCTGGTGAGGGACCTGCC
ACAGGGCTTCTCTGCCTTGGAACCACTGGTGGACCTGCCAATTGGCATCA
ACATCACCAGGTTCCAGACCCTGCTGGCTCTGCACAGGTCCTACCTGACA
CCTGGAGACTCCTCCTCTGGCTGGACAGCAGGAGCAGCAGCCTACTATGT
GGGCTACCTCCAACCAAGGACCTTCCTGCTGAAATACAATGAGAATGGCA
CCATCACAGATGCTGTGGACTGTGCCCTGGACCCACTGTCTGAGACCAAG
TGTACCCTGAAATCCTTCACAGTGGAGAAGGGCATCTACCAGACCAGCAA
CTTCAGGGTCCAACCAACAGAGAGCATTGTGAGGTTTCCAAACATCACCA
ACCTGTGTCCATTTGGAGAGGTGTTCAATGCCACCAGGTTTGCCTCTGTC
TATGCCTGGAACAGGAAGAGGATTAGCAACTGTGTGGCTGACTACTCTGT
GCTCTACAACTCTGCCTCCTTCAGCACCTTCAAGTGTTATGGAGTGAGCC
CAACCAAACTGAATGACCTGTGTTTCACCAATGTCTATGCTGACTCCTTT
GTGATTAGGGGAGATGAGGTGAGACAGATTGCCCCTGGACAAACAGGCAA
GATTGCTGACTACAACTACAAACTGCCTGATGACTTCACAGGCTGTGTGA
TTGCCTGGAACAGCAACAACCTGGACAGCAAGGTGGGAGGCAACTACAAC
TACCTCTACAGACTGTTCAGGAAGAGCAACCTGAAACCATTTGAGAGGGA
CATCAGCACAGAGATTTACCAGGCTGGCAGCACACCATGTAATGGAGTGG
AGGGCTTCAACTGTTACTTTCCACTCCAATCCTATGGCTTCCAACCAACC
AATGGAGTGGGCTACCAACCATACAGGGTGGTGGTGCTGTCCTTTGAACT
GCTCCATGCCCCTGCCACAGTGTGTGGACCAAAGAAGAGCACCAACCTGG
TGAAGAACAAGTGTGTGAACTTCAACTTCAATGGACTGACAGGCACAGGA
GTGCTGACAGAGAGCAACAAGAAGTTCCTGCCATTCCAACAGTTTGGCAG
GGACATTGCTGACACCACAGATGCTGTGAGGGACCCACAGACCTTGGAGA
TTCTGGACATCACACCATGTTCCTTTGGAGGAGTGTCTGTGATTACACCT
GGCACCAACACCAGCAACCAGGTGGCTGTGCTCTACCAGGGTGTGAACTG
TACTGAGGTGCCTGTGGCTATCCATGCTGACCAACTTACACCAACCTGGA
GGGTCTACAGCACAGGCAGCAATGTGTTCCAGACCAGGGCTGGCTGTCTG
ATTGGAGCAGAGCATGTGAACAACTCCTATGAGTGTGACATCCCAATTGG
AGCAGGCATCTGTGCCTCCTACCAGACCCAGACCAACAGCCCAAGGAGGG
CAAGGTCTGTGGCAAGCCAGAGCATCATTGCCTACACAATGAGTCTGGGA
GCAGAGAACTCTGTGGCTTACAGCAACAACAGCATTGCCATCCCAACCAA
CTTCACCATCTCTGTGACCACAGAGATTCTGCCTGTGAGTATGACCAAGA
CCTCTGTGGACTGTACAATGTATATCTGTGGAGACAGCACAGAGTGTAGC
AACCTGCTGCTCCAATATGGCTCCTTCTGTACCCAACTTAACAGGGCTCT
GACAGGCATTGCTGTGGAACAGGACAAGAACACCCAGGAGGTGTTTGCCC
AGGTGAAGCAGATTTACAAGACACCTCCAATCAAGGACTTTGGAGGCTTC
AACTTCAGCCAGATTCTGCCTGACCCAAGCAAGCCAAGCAAGAGGTCCTT
CATTGAGGACCTGCTGTTCAACAAGGTGACCCTGGCTGATGCTGGCTTCA
TCAAGCAATATGGAGACTGTCTGGGAGACATTGCTGCCAGGGACCTGATT
TGTGCCCAGAAGTTCAATGGACTGACAGTGCTGCCTCCACTGCTGACAGA
TGAGATGATTGCCCAATACACCTCTGCCCTGCTGGCTGGCACCATCACCT
CTGGCTGGACCTTTGGAGCAGGAGCAGCCCTCCAAATCCCATTTGCTATG
CAGATGGCTTACAGGTTCAATGGCATTGGAGTGACCCAGAATGTGCTCTA
TGAGAACCAGAAACTGATTGCCAACCAGTTCAACTCTGCCATTGGCAAGA
TTCAGGACTCCCTGTCCAGCACAGCCTCTGCCCTGGGCAAACTCCAAGAT
GTGGTGAACCAGAATGCCCAGGCTCTGAACACCCTGGTGAAGCAACTTTC
CAGCAACTTTGGAGCCATCTCCTCTGTGCTGAATGACATCCTGAGCAGAC
TGGACAAGGTGGAGGCTGAGGTCCAGATTGACAGACTGATTACAGGCAGA
CTCCAATCCCTCCAAACCTATGTGACCCAACAACTTATCAGGGCTGCTGA
GATTAGGGCATCTGCCAACCTGGCTGCCACCAAGATGAGTGAGTGTGTGC
TGGGACAAAGCAAGAGGGTGGACTTCTGTGGCAAGGGCTACCACCTGATG
AGTTTTCCACAGTCTGCCCCTCATGGAGTGGTGTTCCTGCATGTGACCTA
TGTGCCTGCCCAGGAGAAGAACTTCACCACAGCCCCTGCCATCTGCCATG
ATGGCAAGGCTCACTTTCCAAGGGAGGGAGTGTTTGTGAGCAATGGCACC
CACTGGTTTGTGACCCAGAGGAACTTCTATGAACCACAGATTATCACCAC
AGACAACACCTTTGTGTCTGGCAACTGTGATGTGGTGATTGGCATTGTGA
ACAACACAGTCTATGACCCACTCCAACCTGAACTGGACTCCTTCAAGGAG
GAACTGGACAAATACTTCAAGAACCACACCAGCCCTGATGTGGACCTGGG
AGACATCTCTGGCATCAATGCCTCTGTGGTGAACATCCAGAAGGAGATTG
ACAGACTGAATGAGGTGGCTAAGAACCTGAATGAGTCCCTGATTGACCTC
CAAGAACTGGGCAAATATGAACAATACATCAAGTGGCCATGGTACATCTG
GCTGGGCTTCATTGCTGGACTGATTGCCATTGTGATGGTGACCATAATGC
TGTGTTGTATGACCTCCTGTTGTTCCTGTCTGAAAGGCTGTTGTTCCTGT
GGCTCCTGTTGTAAGTTTGATGAGGATGACTCTGAACCTGTGCTGAAAGG
AGTGAAACTGCACTACACC
Human CXCL9
[SEQ ID NO: 2]
ATGAAGAAAAGTGGTGTTCTTTTCCTCTTGGGCATCATCTTGCTGGTTCT
GATTGGAGTGCAAGGAACCCCAGTAGTGAGAAAGGGTCGCTGTTCCTGCA
TCAGCACCAACCAAGGGACTATCCACCTACAATCCTTGAAAGACCTTAAA
CAATTTGCCCCAAGCCCTTCCTGCGAGAAAATTGAAATCATTGCTACACT
GAAGAATGGAGTTCAAACATGTCTAAACCCAGATTCAGCAGATGTGAAGG
AACTGATTAAAAAGTGGGAGAAACAGGTCAGCCAAAAGAAAAAGCAAAAG
AATGGGAAAAAACATCAAAAAAAGAAAGTTCTGAAAGTTCGAAAATCTCA
ACGTTCTCGTCAAAAGAAGACTACATAA
Human IL-7
[SEQ ID NO: 3]
ATGTTCCATGTTTCTTTTAGGTATATCTTTGGACTTCCTCCCCTGATCCT
TGTTCTGTTGCCAGTAGCATCATCTGATTGTGATATTGAAGGTAAAGATG
GCAAACAATATGAGAGTGTTCTAATGGTCAGCATCGATCAATTATTGGAC
AGCATGAAAGAAATTGGTAGCAATTGCCTGAATAATGAATTTAACTTTTT
TAAAAGACATATCTGTGATGCTAATAAGGAAGGTATGTTTTTATTCCGTG
CTGCTCGCAAGTTGAGGCAATTTCTTAAAATGAATAGCACTGGTGATTTT
GATCTCCACTTATTAAAAGTTTCAGAAGGCACAACAATACTGTTGAACTG
CACTGGCCAGGTTAAAGGAAGAAAACCAGCTGCCCTGGGTGAAGCCCAAC
CAACAAAGAGTTTGGAAGAAAATAAATCTTTAAAGGAACAGAAAAAACTG
AATGACTTGTGTTTCCTAAAGAGACTATTACAAGAGATAAAAACTTGTTG
GAATAAAATTTTGATGGGCACTAAAGAACACTGA
P2A sequence
[SEQ ID NO: 4]
GSGATNFSLLKQAGDVEENPGP
P2A
[SEQ ID NO: 5]
GGATCCGGAGCAACAAACTTCTCACTGCTGAAACAGGCCGGAGATGTGGA
GGAAAATCCAGGGCCC
S 5′ primer
[SEQ ID NO: 6]
CGGGTACCGCCACCATGTTTGTGTTCCTGGTGCTGCTGCCACTGGTGTCC
S 3′ primer-1
[SEQ ID NO: 7]
GAAGTTTGTTGCTCCGGATCCGGTGTAGTGCAGTTTCACTCCTTTCAGCA
CAGG
S 3′ primer-2
[SEQ ID NO: 8]
GGGCCCTGGATTTTCCTCCACATCTCCGGCCTGTTTCAGCAGTGAGAAGT
TTGTTGCTCCGGATCC
S-P2A 5′ primer
[SEQ ID NO: 9]
CTGAACCTGTGCTGAAAGGAGTGAAACTGCACTACACCGGATCCGGAGCA
ACAAACTTC
CXCL9 5′ primer
[SEQ ID NO: 10]
GGATCCGGAGCAACAAACTTCTCACTGCTGAAACAGGCCGGAGATGTGGA
GGAAAATCCAGGGCCCATGAAGAAAAGTGGTGTTCTTTTCCTCTTG
CXCL9 3′ primer
[SEQ ID NO: 11]
GCTCTAGATTATGTAGTCTTCTTTTGACGAGAACG
IL-7 5′ primer
[SEQ ID NO: 12]
GGATCCGGAGCAACAAACTTCTCACTGCTGAAACAGGCCGGAGATGTGGA
GGAAAATCCAGGGCCCATGTTCCATGTTTCTTTTAGGTATATCTTTGG
IL-7 3′ primer
[SEQ ID NO: 13]
GCTCTAGATCAGTGTTCTTTAGTGCCCATC
2P substitution 5′ primer
[SEQ ID NO: 14]
CAGACTGGACCCGCCGGAGGCTGAGG
2P substitution 3′ primer
[SEQ ID NO: 15]
CTCAGGATGTCATTCAGC

1-19. (canceled)

20. A recombinant expression vector comprising:
a gene sequence encoding a coronavirus surface spike protein (S protein) comprising a mutation at amino acid position 614 of the spike protein;
an adjuvant gene sequence; and
a gene sequence encoding a P2A peptide.

21. The recombinant expression vector according to claim 20, wherein the gene sequence encoding the coronavirus surface spike protein (S protein) comprising the mutation at amino acid position 614 of the spike protein is set forth in SEQ ID NO: 1.

22. The recombinant expression vector according to claim 20, wherein the mutation is an aspartic acid (D)-to-glycine (G) substitution.

23. The recombinant expression vector according to claim 20, wherein the adjuvant gene sequence is a chemokine (C-X-C motif) ligand 9 (CXCL9) gene, or an interleukin 7 (IL-7) gene.

24. The recombinant expression vector according to claim 23, wherein the chemokine (C-X-C motif) ligand 9 gene is set forth in SEQ ID NO: 2.

25. The recombinant expression vector according to claim 23, wherein the interleukin 7 gene is set forth in SEQ ID NO: 3.

26. The recombinant expression vector according to claim 20, wherein the gene encoding a P2A peptide is set forth in SEQ ID NO: 5.

27. A recombinant transformant transformed with the recombinant expression vector according to claim 20.

28. The recombinant transformant according to claim 27, wherein the transformant is selected from the group consisting of a microorganism, a cell, an animal, a plant, and a virus.

29. The recombinant transformant according to claim 28, wherein the virus is an adenovirus.

30. The recombinant transformant according to claim 29, wherein the adenovirus is adenovirus type 5.

31. A vaccine composition for preventing a coronavirus disease (COVID-19) comprising a transformant transformed with the recombinant expression vector according to claim 27.

32. The vaccine composition according to claim 31, wherein the transformant expresses a SARS-CoV-2 recombinant protein.

33. The vaccine composition according to claim 31, wherein the vaccine composition is administered intramuscularly, administered intranasally, or inhaled intranasally.

34. The vaccine composition according to claim 31, wherein the coronavirus disease is at least one selected from the group consisting of human coronavirus 229E (HCoV-229E), human coronavirus OC43 (HCoV-OC43), human coronavirus HKU1 (HCoV-HKU1), human coronavirus NL63 (HCoV-NL63), severe acute respiratory syndrome coronavirus (SARS-CoV), severe acute respiratory syndrome virus-2 (SARS-CoV-2), Middle East respiratory syndrome coronavirus (MERS-CoV), porcine epidemic diarrhea virus (PEDV), transmissible gastroenteritis virus (TGEV), porcine hemagglutinating encephalomyelitis virus (PHEV), bovine coronavirus (BCoV), equine coronavirus (EqCoV), murine coronavirus (MuCoV), canine coronavirus (CCoV), feline coronavirus (FCoV), Miniopterus bat coronavirus-1, Miniopterus bat coronavirus HKU8, Rhinolophus bat coronavirus HKU2, Scotophilus bat coronavirus 512, Tylonycteris bat coronavirus HKU4, Pipistrellus bat coronavirus HKU5, Rousettus bat coronavirus HKU9, avian coronavirus, Beluga whale coronavirus SW1, Bulbul coronavirus HKU11, Thrush coronavirus HKU12, and Munia coronavirus HKU13.

35. A coronavirus disease (COVID-19) prime-boost vaccine composition comprising, as an active ingredient, a recombinant adenovirus obtained by transfecting an adenovirus with the recombinant expression vector according to claim 20 and culturing the transfected adenovirus.

36. A pharmaceutical composition for preventing or treating a coronavirus disease (COVID-19) comprising, as an active ingredient, a recombinant adenovirus obtained by transfecting an adenovirus with the recombinant expression vector according to claim 20 and culturing the transfected adenovirus.

37. A method for preventing or treating coronavirus disease (COVID-19) comprising a step of administering a recombinant adenovirus obtained by transfecting an adenovirus with the recombinant expression vector according to claim 20 and culturing the transfected adenovirus.

38. Use of a recombinant adenovirus, obtained by transfecting an adenovirus with the recombinant expression vector according to claim 20 and culturing the transfected adenovirus, as an active ingredient, for preventing or treating a coronavirus disease (COVID-19).


SEQUENCE LISTING (15 sequences)

SEQ ID NO: 1 - DNA, 3819 bp, synthetic construct
  source 1..3819: mol_type = other DNA
atgtttgtgttcctggtgctgctgccactggtgtccagccagtgtgtgaacctgaccaccaggacccaacttcctcctgcctacaccaactccttcaccaggggagtctactaccctgacaaggtgttcaggtcctctgtgctgcacagcacccaggacctgttcctgccattcttcagcaatgtgacctggttccatgccatccatgtgtctggcaccaatggcaccaagaggtttgacaaccctgtgctgccattcaatgatggagtctactttgccagcacagagaagagcaacatcatcaggggctggatttttggcaccaccctggacagcaagacccagtccctgctgattgtgaacaatgccaccaatgtggtgattaaggtgtgtgagttccagttctgtaatgacccattcctgggagtctactaccacaagaacaacaagtcctggatggagtctgagttcagggtctactcctctgccaacaactgtacctttgaatatgtgagccaaccattcctgatggacttggagggcaagcagggcaacttcaagaacctgagggagtttgtgttcaagaacattgatggctacttcaagatttacagcaaacacacaccaatcaacctggtgagggacctgccacagggcttctctgccttggaaccactggtggacctgccaattggcatcaacatcaccaggttccagaccctgctggctctgcacaggtcctacctgacacctggagactcctcctctggctggacagcaggagcagcagcctactatgtgggctacctccaaccaaggaccttcctgctgaaatacaatgagaatggcaccatcacagatgctgtggactgtgccctggacccactgtctgagaccaagtgtaccctgaaatccttcacagtggagaagggcatctaccagaccagcaacttcagggtccaaccaacagagagcattgtgaggtttccaaacatcaccaacctgtgtccatttggagaggtgttcaatgccaccaggtttgcctctgtctatgcctggaacaggaagaggattagcaactgtgtggctgactactctgtgctctacaactctgcctccttcagcaccttcaagtgttatggagtgagcccaaccaaactgaatgacctgtgtttcaccaatgtctatgctgactcctttgtgattaggggagatgaggtgagacagattgcccctggacaaacaggcaagattgctgactacaactacaaactgcctgatgacttcacaggctgtgtgattgcctggaacagcaacaacctggacagcaaggtgggaggcaactacaactacctctacagactgttcaggaagagcaacctgaaaccatttgagagggacatcagcacagagatttaccaggctggcagcacaccatgtaatggagtggagggcttcaactgttactttccactccaatcctatggcttccaaccaaccaatggagtgggctaccaaccatacagggtggtggtgctgtcctttgaactgctccatgcccctgccacagtgtgtggaccaaagaagagcaccaacctggtgaagaacaagtgtgtgaacttcaacttcaatggactgacaggcacaggagtgctgacagagagcaacaagaagttcctgccattccaacagtttggcagggacattgctgacaccacagatgctgtgagggacccacagaccttggagattctggacatcacaccatgttcctttggaggagtgtctgtgattacacctggcaccaacaccagcaaccaggtggctgtgctctaccagggtgtgaactgtactgaggtgcctgtggctatccatgctgaccaacttacaccaacctggagggtctacagcacaggcagcaatgtgttccagaccagggctggctgtctgattggagcagagcatgtgaacaactcctatgagtgtgacatcccaattggagcaggcatctgtgcctcctaccagacccagaccaacagcccaaggagggcaaggtctgtggcaagccagagcatcattgcctacacaatgagtctgggagcagagaactctgtggcttacagcaacaacagcattgccatcccaaccaacttcaccatctctgtgaccacagagattctgcctgtgagtatgaccaagacctctgtggactgtacaatgtatatctgtggagacagcacagagtgtagcaacctgctgctccaatatggctccttctgtacccaacttaacagggctctgacaggcattgctgtggaacaggacaagaacacccaggaggtgtttgcccaggtgaagcagatttacaagacacctccaatcaaggactttggaggcttcaacttcagccagattctgcctgacccaagcaagccaagcaagaggtccttcattgaggacctgctgttcaacaaggtgaccctggctgatgctggcttcatcaagcaatatggagactgtctgggagacattgctgccagggacctgatttgtgcccagaagttcaatggactgacagtgctgcctccactgctgacagatgagatgattgcccaatacacctctgccctgctggctggcaccatcacctctggctggacctttggagcaggagcagccctccaaatcccatttgctatgcagatggcttacaggttcaatggcattggagtgacccagaatgtgctctatgagaaccagaaactgattgccaaccagttcaactctgccattggcaagattcaggactccctgtccagcacagcctctgccctgggcaaactccaagatgtggtgaaccagaatgcccaggctctgaacaccctggtgaagcaactttccagcaactttggagccatctcctctgtgctgaatgacatcctgagcagactggacaaggtggaggctgaggtccagattgacagactgattacaggcagactccaatccctccaaacctatgtgacccaacaacttatcagggctgctgagattagggcatctgccaacctggctgccaccaagatgagtgagtgtgtgctgggacaaagcaagagggtggacttctgtggcaagggctaccacctgatgagttttccacagtctgcccctcatggagtggtgttcctgcatgtgacctatgtgcctgcccaggagaagaacttcaccacagcccctgccatctgccatgatggcaaggctcactttccaagggagggagtgtttgtgagcaatggcacccactggtttgtgacccagaggaacttctatgaaccacagattatcaccacagacaacacctttgtgtctggcaactgtgatgtggtgattggcattgtgaacaacacagtctatgacccactccaacctgaactggactccttcaaggaggaactggacaaatacttcaagaaccacaccagccctgatgtggacctgggagacatctctggcatcaatgcctctgtggtgaacatccagaaggagattgacagactgaatgaggtggctaagaacctgaatgagtccctgattgacctccaagaactgggcaaatatgaacaatacatcaagtggccatggtacatctggctgggcttcattgctggactgattgccattgtgatggtgaccataatgctgtgttgtatgacctcctgttgttcctgtctgaaaggctgttgttcctgtggctcctgttgtaagtttgatgaggatgactctgaacctgtgctgaaaggagtgaaactgcactacacc

SEQ ID NO: 2 - DNA, 378 bp, Homo sapiens
  source 1..378: mol_type = genomic DNA
atgaagaaaagtggtgttcttttcctcttgggcatcatcttgctggttctgattggagtgcaaggaaccccagtagtgagaaagggtcgctgttcctgcatcagcaccaaccaagggactatccacctacaatccttgaaagaccttaaacaatttgccccaagcccttcctgcgagaaaattgaaatcattgctacactgaagaatggagttcaaacatgtctaaacccagattcagcagatgtgaaggaactgattaaaaagtgggagaaacaggtcagccaaaagaaaaagcaaaagaatgggaaaaaacatcaaaaaaagaaagttctgaaagttcgaaaatctcaacgttctcgtcaaaagaagactacataa

SEQ ID NO: 3 - DNA, 534 bp, Homo sapiens
  source 1..534: mol_type = genomic DNA
atgttccatgtttcttttaggtatatctttggacttcctcccctgatccttgttctgttgccagtagcatcatctgattgtgatattgaaggtaaagatggcaaacaatatgagagtgttctaatggtcagcatcgatcaattattggacagcatgaaagaaattggtagcaattgcctgaataatgaatttaacttttttaaaagacatatctgtgatgctaataaggaaggtatgtttttattccgtgctgctcgcaagttgaggcaatttcttaaaatgaatagcactggtgattttgatctccacttattaaaagtttcagaaggcacaacaatactgttgaactgcactggccaggttaaaggaagaaaaccagctgccctgggtgaagcccaaccaacaaagagtttggaagaaaataaatctttaaaggaacagaaaaaactgaatgacttgtgtttcctaaagagactattacaagagataaaaacttgttggaataaaattttgatgggcactaaagaacactga

SEQ ID NO: 4 - protein, 22 aa, synthetic construct
GSGATNFSLLKQAGDVEENPGP

SEQ ID NO: 5 - DNA, 66 bp, synthetic construct
  source 1..66: mol_type = other DNA
ggatccggagcaacaaacttctcactgctgaaacaggccggagatgtggaggaaaatccagggccc

SEQ ID NO: 6 - DNA, 50 bp, synthetic construct
  source 1..50: mol_type = other DNA
cgggtaccgccaccatgtttgtgttcctggtgctgctgccactggtgtcc

SEQ ID NO: 7 - DNA, 54 bp, synthetic construct
  source 1..54: mol_type = other DNA
gaagtttgttgctccggatccggtgtagtgcagtttcactcctttcagcacagg

SEQ ID NO: 8 - DNA, 66 bp, synthetic construct
  source 1..66: mol_type = other DNA
gggccctggattttcctccacatctccggcctgtttcagcagtgagaagtttgttgctccggatcc

SEQ ID NO: 9 - DNA, 59 bp, synthetic construct
  source 1..59: mol_type = other DNA
ctgaacctgtgctgaaaggagtgaaactgcactacaccggatccggagcaacaaacttc

SEQ ID NO: 10 - DNA, 96 bp, synthetic construct
  source 1..96: mol_type = other DNA
ggatccggagcaacaaacttctcactgctgaaacaggccggagatgtggaggaaaatccagggcccatgaagaaaagtggtgttcttttcctcttg

SEQ ID NO: 11 - DNA, 35 bp, synthetic construct
  source 1..35: mol_type = other DNA
gctctagattatgtagtcttcttttgacgagaacg

SEQ ID NO: 12 - DNA, 98 bp, synthetic construct
  source 1..98: mol_type = other DNA
ggatccggagcaacaaacttctcactgctgaaacaggccggagatgtggaggaaaatccagggcccatgttccatgtttcttttaggtatatctttgg

SEQ ID NO: 13 - DNA, 30 bp, synthetic construct
  source 1..30: mol_type = other DNA
gctctagatcagtgttctttagtgcccatc

SEQ ID NO: 14 - DNA, 26 bp, synthetic construct
  source 1..26: mol_type = other DNA
cagactggacccgccggaggctgagg

SEQ ID NO: 15 - DNA, 18 bp, synthetic construct
  source 1..18: mol_type = other DNA
ctcaggatgtcattcagc